(19) United States
(12) Patent Application Publication
(10) Pub. No.: US 2022/0403388 A1
(43) Pub. Date: Dec. 22, 2022

(54) Oligonucleotide Progranulin Agonists

(71) Applicant: Hoffmann-La Roche Inc., Little Falls, NJ (US)

(72) Inventors: Lars Joenson, Horsholm (DK); Soren V. Rasmussen, Horsholm (DK); Jesper Worm, Horsholm (DK); Dorthe Vang Larsen, Horsholm (DK); Johannes Braun, Horsholm (DK)

(21) Appl. No.: 17/833,085

(22) Filed: Jun. 6, 2022

(30) Foreign Application Priority Data
Jun. 8, 2021 (EP) 21178235.4

(51) Int. Cl. C12N 15/113

(52) U.S. Cl. CPC C12N 15/113; C12N 2310/3231; C12N 2310/351

(57) ABSTRACT

The present invention relates to oligonucleotides that upregulate or restore the expression of progranulin in cells by targeting the promoter region of the progranulin gene. The invention further relates to pharmaceutical compositions and methods for the treatment of diseases associated with progranulin, specifically progranulin haploinsufficiency and neurological disorders.

### CROSS REFERENCE TO RELATED APPLICATIONS

This application claims benefit of and priority to the European Patent Application EP 21178235.4 filed on Jun. 8, 2021, which is herein incorporated by reference in its entirety where permissible.

### REFERENCE TO SEQUENCE LISTING

The instant application contains a Sequence Listing which has been submitted electronically in ASCII format and is hereby incorporated by reference in its entirety. Said ASCII copy, created on May 26, 2022, is named “P122467US (067211.018US1) Sequence Listing.txt” and is 79,953 bytes in size.

### TECHNICAL FIELD OF THE INVENTION

The present invention relates to oligonucleotides that upregulate or restore the expression of progranulin in cells; conjugates, salts and pharmaceutical compositions thereof; and their use in the treatment of neurological disorders, and disorders associated with progranulin haploinsufficiency.

### BACKGROUND OF THE INVENTION

Progranulin (PGRN) is a highly conserved secreted protein that is expressed in multiple cell types, both in the CNS and in peripheral tissues. Progranulin is the precursor of the granulin polypeptides.

Deficiency of the secreted protein progranulin in the central nervous system causes the neurodegenerative disease frontotemporal dementia (FTD). Pathogenic mutations in the progranulin gene (GRN) lead to a loss of about 50% in progranulin levels through haploinsufficiency and to intraneuronal aggregation of the TAR-DNA binding protein of 43 kDA (TDP-43). Progranulin plays a supportive and protective role in numerous processes within the brain, including neurite outgrowth, synapse biology, response to exogenous stressors, lysosomal function, neuroinflammation, and angiogenesis in both cell autonomous and non-autonomous manners.

TDP-43 is associated with, and implicated in, several neurodegenerative diseases. TDP-43 pathology is associated with cytoplasmic TDP-43 aggregation. For example, more than 95% of amyotrophic lateral sclerosis (ALS) patients display pathological mislocalization of TDP-43 and several mutations in the TARDBP gene cause familial ALS.

The presence of cytoplasmic TDP-43 aggregates is associated with a concomitant loss of nuclear TDP-43, and there is evidence of both loss of function and gain of function associated pathophysiologies.

Both directly and via its conversion to granulins, progranulin regulates lysosomal function, cell growth, survival, repair, and inflammation. Progranulin has a major role in regulation of lysosomal function-associated microglial responses in the CNS. Autosomal dominant mutations of the progranulin (GRN) gene leading to protein haploinsufficiency are linked to familial frontotemporal dementia with neuropathologic frontotemporal lobar degeneration (FTLD) associated with accumulation of TDP-43 inclusions (FTLD-TDP). Homozygous GRN mutations are linked to neuronal ceroid lipofuscinosis (NCL) (Townley, et al., Neurology. 2018 Jun. 12; 90(24): 1127).

Mutations in the progranulin gene (GRN) have recently been identified as a cause of about 5% of all FTD, including some sporadic cases. Recent studies using mouse models have defined the expression of PGRN in the brain (Petkau et al., 2010). PGRN is expressed late in neurodevelopment, localizing with markers of mature neurons. PGRN is expressed in neurons in most brain regions, with highest expression in the thalamus, hippocampus, and cortex. Microglia cells also express progranulin, and the level of expression is unregulated by microglial activation. Around 70 different GRN mutations have been identified in FTD and all reduce progranulin levels or result in loss of progranulin function.

There is therefore an urgent need for therapeutic agents that can increase or restore the expression of progranulin.

### SUMMARY OF THE INVENTION

The invention provides oligonucleotide agonists of progranulin or oligonucleotide progranulin agonists—i.e. oligonucleotides that are complementary to a progranulin nucleic acid sequence. Specifically, the invention provides oligonucleotide progranulin agonists that target the progranulin promoter. These oligonucleotides are capable of up-regulating the expression of the progranulin gene, GRN, and/or progranulin, PGRN. Alternatively stated, the invention provides oligonucleotide positive modulators (i.e. agonists) of progranulin.

The oligonucleotides of the invention may be used to restore progranulin expression in cells, such as cells which exhibit progranulin haploinsufficiency, or to enhance expression of progranulin in cells.

The invention provides oligonucleotide progranulin agonists, wherein the oligonucleotide is 8-40 nucleotides in length and comprises a contiguous nucleotide sequence of 8-40 nucleotides in length which is complementary, such as fully complementary, to the promoter of the human granulin precursor (progranulin) gene.

The human granulin precursor (progranulin) gene may have NCBI Reference Sequence: NG_007886.1.

The invention provides oligonucleotide progranulin agonists, wherein the oligonucleotide is 8-40 nucleotides in length and comprises a contiguous nucleotide sequence of 8-40 nucleotides in length which is complementary, such as fully complementary, to SEQ ID NO: 1.

The invention provides double stranded oligonucleotide progranulin agonists wherein the oligonucleotide is 8-40 nucleotides in length and comprises a contiguous nucleotide sequence of 8-40 nucleotides in length which is complementary, such as fully complementary, to the promoter of the human progranulin gene.

The double stranded oligonucleotide progranulin agonist may be a small activating RNA (saRNA).

The invention provides single stranded oligonucleotide progranulin agonists wherein the oligonucleotide is 8-40 nucleotides in length and comprises a contiguous nucleotide sequence of 8-40 nucleotides in length which is complementary, such as fully complementary, to the promoter of the human progranulin gene.

The single stranded oligonucleotide may be an antisense oligonucleotide. The single stranded oligonucleotide may target either the sense or antisense strand of the human progranulin gene.

The invention provides an oligonucleotide progranulin agonist covalently attached to at least one conjugate moiety.

The invention provides an oligonucleotide progranulin agonist in the form of a pharmaceutically acceptable salt.

The invention provides a pharmaceutical composition comprising an oligonucleotide progranulin agonist and a pharmaceutically acceptable diluent, solvent, carrier, salt and/or adjuvant.

The invention provides an in vivo or in vitro method for upregulating or restoring progranulin expression in a target cell, said method comprising administering an oligonucleotide progranulin agonist, or the pharmaceutical composition, of the invention in an effective amount to the cell.

The invention provides a method for treating or preventing a disease comprising administering a therapeutically or prophylactically effective amount of an oligonucleotide progranulin agonist or a pharmaceutical composition of the invention, to a subject suffering from or susceptible to a disease.

The invention provides an oligonucleotide progranulin agonist, or a pharmaceutical composition of the invention, for use in the treatment or prevention of a disease.

In all aspects of the invention, the disease or disorder to be treated may be a neurological disease, a TDP-43 pathology, progranulin haploinsufficiency, frontotemporal dementia (FTD), amyotrophic lateral sclerosis (ALS), frontotemporal dementia with neuropathologic frontotemporal lobar degeneration (FTLD), familial frontotemporal dementia with neuropathologic frontotemporal lobar degeneration associated with accumulation of TDP-43 inclusions (FTLD-TDP), or neuronal ceroid lipofuscinosis (NCL).

These and other aspects and embodiments of the invention will be described in further detail below.

### BRIEF DESCRIPTION OF THE DRAWINGS

FIG. 1 shows progranulin mRNA expression levels in H4 neuroglioma cells at three days post treatment with saRNA oligonucleotides corresponding to SEQ ID NO: 2, SEQ ID NO: 3, SEQ ID NO: 4, and SEQ ID NO: 5. mRNA expression analysis was performed using ddPCR according to a predesigned qPCR assay, and quantified relative to mock transfected controls.

FIG. 2 shows progranulin mRNA expression levels in H4 neuroglioma cells at three days post treatment with saRNA oligonucleotides corresponding to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, and SEQ ID NO: 25. mRNA expression analysis was performed using ddPCR according to a predesigned qPCR assay, and quantified relative to the housekeeping gene HPRT1.

FIG. 3 shows progranulin protein expression levels in the supernatant of H4 neuroglioma cells at three days post treatment with saRNA oligonucleotides corresponding to SEQ ID NO: 9, SEQ ID NO: 11, SEQ ID NO: 17, and SEQ ID NO: 18. Progranulin protein was quantified in the culture media using a human progranulin ELISA kit (Abcam; ab252364), and reported relative to mock transfected controls.

FIG. 4 is a sashimi plot showing number of reads spanning each annotated exons of GRN and shows that saRNAs corresponding to SEQ ID NO: 9, SEQ ID NO: 11, SEQ ID NO: 17, and SEQ ID NO: 18 targeting the progranulin promoter show upregulation of mature GRN mRNA.

FIG. 5 shows that mutations in the seed region of the antisense strand SEQ ID NO: 80 (i.e. saRNA formed from SEQ ID NO: 80 and SEQ ID NO: 83) and SEQ ID NO: 81 (i.e. saRNA formed from SEQ ID NO: 81 and SEQ ID NO: 84) abolishes the dose dependent upregulation of GRN mRNA.

### DETAILED DESCRIPTION OF THE INVENTION

I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, the terms “treat,” “treating,” “treatment” and “therapeutic use” refer to the elimination, reduction or amelioration of one or more symptoms of a disease or disorder. Specifically, the term “treatment” may refer to both treatment of an existing disease (e.g. a disease or disorder as herein referred to), or prevention of a disease (i.e. prophylaxis). It will therefore be recognized that treatment as referred to herein may, in some embodiments, be prophylactic. As used herein, a “therapeutically effective amount” refers to that amount of a therapeutic agent sufficient to mediate a clinically relevant elimination, reduction or amelioration of such symptoms. An effect is clinically relevant if its magnitude is sufficient to impact the health or prognosis of a recipient subject. A therapeutically effective amount may refer to the amount of therapeutic agent sufficient to delay or minimize the onset of disease. A therapeutically effective amount may also refer to the amount of the therapeutic agent that provides a therapeutic benefit in the treatment or management of a disease.

II. Progranulin Agonist

The term “progranulin agonist” as used herein refers to a compound, in this case an oligonucleotide, which is capable of enhancing the expression of progranulin mRNA transcripts and/or progranulin protein in a cell, such as a cell which is expressing progranulin.

In certain embodiments the oligonucleotide progranulin agonists of the present invention may enhance the production of progranulin mRNA and/or progranulin protein by at least about 10%. In other embodiments the oligonucleotide progranulin agonists of the present invention may enhance the production of progranulin mRNA and/or progranulin protein by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600% or more.

The inventors have identified that the expression level of the progranulin mRNA transcript, and/or the expression level of encoded protein products, can be effectively enhanced by targeting the promotor region of the progranulin gene using oligonucleotides, particularly double stranded oligonucleotides such as short activating RNAs (saRNAs) or single stranded antisense oligonucleotides.

Described herein are target sites present on the human progranulin gene, particularly within the promoter region, which can be targeted by the oligonucleotides of the invention. The oligonucleotides of the invention are agonists of progranulin, i.e. they increase production of progranulin mRNA and/or protein.

The invention provides oligonucleotide progranulin agonists, wherein the oligonucleotide is 8-40 nucleotides in length and comprises a contiguous nucleotide sequence of 8-40 nucleotides in length which is complementary, such as fully complementary, to the promoter of the human granulin precursor (progranulin) gene, NCBI Reference Sequence: NG_007886.1.

The invention relates to oligonucleotide progranulin agonists.

The oligonucleotides of the present invention are progranulin agonists, i.e. they enhance the expression of progranulin. This can mean an increase in the expression of progranulin nucleic acids, such as progranulin mRNA, and/or an increase in the expression of progranulin protein. Enhanced progranulin expression is desirable to treat a range of neurological disorders, such as TDP-43 pathologies, or disorders which are characterized by or caused by progranulin haploinsufficiency.

III. Oligonucleotide

The term “oligonucleotide” as used herein is defined, as is generally understood by the skilled person, as a molecule comprising two or more covalently linked nucleosides. Such covalently bound nucleosides may also be referred to as nucleic acid molecules or oligomers.

Oligonucleotides are commonly made in a laboratory by solid-phase chemical synthesis followed by purification and isolation. When referring to the sequence of an oligonucleotide, reference is made to the sequence or order of nucleobase moieties, or modifications thereof, of the covalently linked nucleotides or nucleosides. The oligonucleotides of the invention are man-made, and are chemically synthesized, and are typically purified or isolated. The oligonucleotides of the invention may comprise one or more modified nucleosides such as 2′ sugar modified nucleosides. The oligonucleotides of the invention may comprise one or more modified internucleoside linkages, such as one or more phosphorothioate internucleoside linkages.

In some embodiments, the oligonucleotide progranulin agonists of the invention are double stranded oligonucleotides.

In some embodiments, the oligonucleotide progranulin agonists of the invention are single stranded oligonucleotides.

In some embodiments, the oligonucleotide progranulin agonists of the invention are 8-40 nucleotides in length.

In some embodiments, the oligonucleotide progranulin agonists of the invention are 8-40 nucleotides in length and comprise a contiguous nucleotide sequence of 8-40 nucleotides.

In some embodiments, the oligonucleotide progranulin agonists of the invention are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments the oligonucleotide progranulin agonists of the invention are at least 12 nucleotides in length.

In some embodiments the oligonucleotide progranulin agonists of the invention are at least 14 nucleotides in length.

In some embodiments the oligonucleotide progranulin agonists of the invention are at least 16 nucleotides in length.

In some embodiments the oligonucleotide progranulin agonists of the invention are at least 18 nucleotides in length.

In some embodiments, the oligonucleotide progranulin agonists of the invention are 21 nucleotides in length.

IV. Contiguous Nucleotide Sequence

The term “contiguous nucleotide sequence” refers to the region of the oligonucleotide which is complementary to a target nucleic acid, which may be or may comprise an oligonucleotide motif sequence. The term is used interchangeably herein with the term “contiguous nucleobase sequence”.

In some embodiments the oligonucleotide comprises the contiguous nucleotide sequence, and may optionally comprise further nucleotide(s), for example a nucleotide linker region which may be used to attach a functional group (e.g. a conjugate group) to the contiguous nucleotide sequence. The nucleotide linker region may or may not be complementary to the target nucleic acid.

It is understood that the contiguous nucleotide sequence of the oligonucleotide cannot be longer than the oligonucleotide as such and that the oligonucleotide cannot be shorter than the contiguous nucleotide sequence.

In some embodiments all the nucleosides of the oligonucleotide constitute the contiguous nucleotide sequence.

The contiguous nucleotide sequence is the sequence of nucleotides in the oligonucleotide of the invention which are complementary to, and in some instances fully complementary to, the target nucleic acid, target sequence, or target site sequence.

In some embodiments, the contiguous nucleotide sequence is 8-40 nucleotides in length.

In some embodiments, the contiguous nucleotide sequence is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments the contiguous nucleotide sequence is at least 12 nucleotides in length.

In some embodiments the contiguous nucleotide sequence is at least 14 nucleotides in length.

In some embodiments the contiguous nucleotide sequence is at least 16 nucleotides in length.

In some embodiments the contiguous nucleotide sequence is at least 18 nucleotides in length.

In some embodiments, the contiguous nucleotide sequence is 19 nucleotides in length.

In some embodiments, the contiguous nucleotide sequence is 20 nucleotides in length.

In some embodiments, the contiguous nucleotide sequence is 21 nucleotides in length.

In some embodiments, the contiguous nucleotide sequence is 22 nucleotides in length.

In some embodiments, the contiguous nucleotide sequence is the same length as the oligonucleotide progranulin agonist.

In some embodiments the oligonucleotide consists of the contiguous nucleotide sequence.

In some embodiments the oligonucleotide is the contiguous nucleotide sequence

V. Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides, comprise a ribose sugar moiety, a nucleobase moiety and one or more phosphate groups (which is absent in nucleosides). Nucleosides and nucleotides may also interchangeably be referred to as “units” or “monomers”.

VI. Modified Nucleoside

Advantageously, the oligonucleotide progranulin agonist of the invention may comprise one or more modified nucleosides.

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. Advantageously, one or more of the modified nucleosides of the oligonucleotides of the invention may comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing. Exemplary modified nucleosides which may be used in the oligonucleotide progranulin agonists of the invention include LNA, 2′-O-MOE, 2′oMe and morpholino nucleoside analogues.

VII. Modified Internucleoside Linkage

Advantageously, the oligonucleotide progranulin agonist of the invention comprises one or more modified internucleoside linkage.

The term “modified internucleoside linkage” is defined as generally understood by the skilled person as linkages other than phosphodiester (PO) linkages, that covalently couple two nucleosides together. The oligonucleotide progranulin agonists of the invention may therefore comprise one or more modified internucleoside linkages such as one or more phosphorothioate internucleoside linkage.

In some embodiments at least 50% of the internucleoside linkages in the oligonucleotide progranulin agonist, or contiguous nucleotide sequence thereof, are phosphorothioate, such as at least 60%, such as at least 70%, such as at least 75%, such as at least 80%, such as at least 90% or more of the internucleoside linkages in the oligonucleotide progranulin agonist, or contiguous nucleotide sequence thereof, are phosphorothioate. In some embodiments all of the internucleoside linkages of the oligonucleotide progranulin agonist, or contiguous nucleotide sequence thereof, are phosphorothioate.

In a further embodiment, the oligonucleotide progranulin agonist comprises at least one modified internucleoside linkage. It is advantageous if at least 75%, such as all, of the internucleoside linkages within the contiguous nucleotide sequence are phosphorothioate or boranophosphate internucleoside linkages.

Advantageously, all the internucleoside linkages of the contiguous nucleotide sequence of the oligonucleotide progranulin agonist may be phosphorothioate, or all the intemucleoside linkages of the oligonucleotide progranulin agonist may be phosphorothioate linkages.

VIII. Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but which are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

IX. Modified Oligonucleotide

The oligonucleotide progranulin agonist of the invention may be a modified oligonucleotide.

The term modified oligonucleotide describes an oligonucleotide comprising one or more sugar-modified nucleosides and/or modified internucleoside linkages. The term “chimeric oligonucleotide” is a term that has been used in the literature to describe oligonucleotides comprising sugar modified nucleosides and DNA nucleosides. In some embodiments, it may be advantageous for the oligonucleotide progranulin agonist of the invention to be a chimeric oligonucleotide.

In some embodiments, the oligonucleotide progranulin agonist or contiguous nucleotide sequence thereof may include modified nucleobases which function as the shown nucleobase in base pairing, for example 5-methyl cytosine may be used in place of methyl cytosine. Inosine may be used as a universal base.

It is understood that the contiguous nucleobase sequences (motif sequence) can be modified to, for example, increase nuclease resistance and/or binding affinity to the target nucleic acid.

The pattern in which the modified nucleosides (such as high affinity modified nucleosides) are incorporated into the oligonucleotide sequence is generally termed oligonucleotide design.

The oligonucleotide progranulin agonists of the invention are designed with modified nucleosides and DNA nucleosides. Advantageously, high affinity modified nucleosides are used.

In an embodiment, the oligonucleotide progranulin agonist comprises at least 1 modified nucleoside, such as at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21, modified nucleosides.

Suitable modifications are described herein under the headings “modified nucleoside”, “high affinity modified nucleosides”, “sugar modifications”, “2′ sugar modifications” and Locked nucleic acids (LNA)”.

X. High Affinity Modified Nucleosides

A high affinity modified nucleoside is a modified nucleotide which, when incorporated into an oligonucleotide enhances the affinity of the oligonucleotide for its complementary target, for example as measured by the melting temperature (T^{m}). A high affinity modified nucleoside of the present invention preferably results in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

XI. Sugar Modifications

The oligonucleotide progranulin agonist of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

XII. 2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA (2′oMe), 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

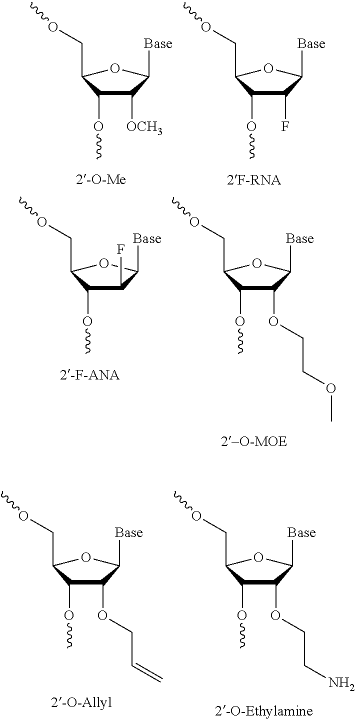

In relation to the present invention 2′ substituted sugar modified nucleosides does not include 2′ bridged nucleosides like LNA.

In an embodiment, the oligonucleotide progranulin agonist comprises one or more sugar modified nucleosides, such as 2′ sugar modified nucleosides. Preferably the oligonucleotide progranulin agonist of the invention comprises one or more 2′ sugar modified nucleoside independently selected from the group consisting of 2′-O-alkyl-RNA, 2′-O-methyl-RNA (2′oMe), 2′-alkoxy-RNA, 2′-O-methoxyethyl-RNA (2′MOE), 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

XIII. Locked Nucleic Acid Nucleosides (LNA Nucleoside)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352 , WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

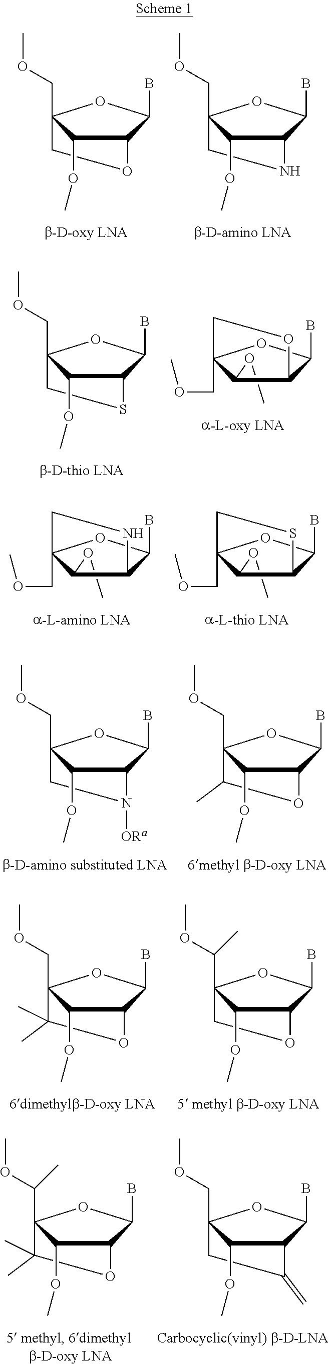

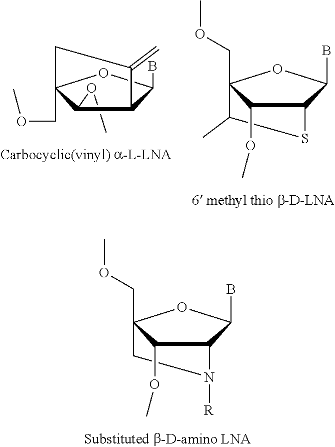

Particular LNA nucleosides are beta-D-oxy-LNA, 6′-methyl-beta-D-oxy LNA such as (S)-6′-methyl-beta-D-oxy-LNA (ScET) and ENA.

A particularly advantageous LNA is beta-D-oxy-LNA.

XIV. Morpholino Oligonucleotides

In some embodiments, the oligonucleotide progranulin agonist of the invention comprises or consists of morpholino nucleosides (i.e. is a Morpholino oligomer and as a phosphorodiamidate Morpholino oligomer (PMO)). Splice modulating morpholino oligonucleotides have been approved for clinical use—see for example eteplirsen, a 30 nt morpholino oligonucleotide targeting a frame shift mutation in DMD, used to treat Duchenne muscular dystrophy. Morpholino oligonucleotides have nucleobases attached to six membered morpholine rings rather ribose, such as methylenemorpholine rings linked through phosphorodiamidate groups, for example as illustrated by the following illustration of 4 consecutive morpholino nucleotides:

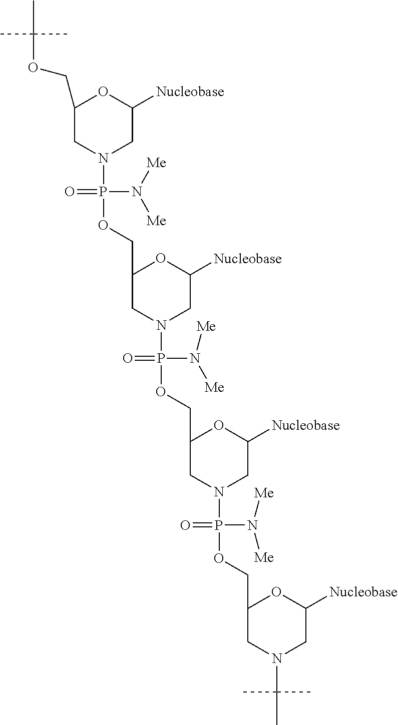

In some embodiments, morpholino oligonucleotides of the invention may be, for example 20-40 morpholino nucleotides in length, such as morpholino 25-35 nucleotides in length.

XV. RNase H Activity and Recruitment

The RNase H activity of an oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10%, at least 20% or more than 20%, of the initial rate determined when using an oligonucleotide having the same base sequence as the modified oligonucleotide being tested, but containing only DNA monomers with phosphorothioate linkages between all monomers in the oligonucleotide, and using the methodology provided by Examples 91-95 of WO01/23613 (hereby incorporated by reference). For use in determining RHase H activity, recombinant RNase H1 is available from Lubio Science GmbH, Lucerne, Switzerland.

DNA oligonucleotides are known to effectively recruit RNaseH, as are gapmer oligonucleotides which comprise a region of DNA nucleosides (typically at least 5 or 6 contiguous DNA nucleosides), flanked 5′ and 3′ by regions comprising 2′ sugar modified nucleosides, typically high affinity 2′ sugar modified nucleosides, such as 2-O-MOE and/or LNA. For effective modulation of splicing, degradation of the pre-mRNA is not desirable, and as such it is preferable to avoid the RNaseH degradation of the target. Therefore, the oligonucleotide progranulin agonists of the invention are not RNaseH recruiting gapmer oligonucleotide.

RNaseH recruitment may be avoided by limiting the number of contiguous DNA nucleotides in the oligonucleotide—therefore mixmer and totalmer designs may be used. Advantageously the oligonucleotide progranulin agonist of the invention, or the contiguous nucleotide sequence thereof, do not comprise more than 3 contiguous DNA nucleosides. Further, advantageously the oligonucleotide progranulin agonists of the invention, or the contiguous nucleotide sequence thereof, do not comprise more than 4 contiguous DNA nucleosides. Further, advantageously, the oligonucleotide progranulin agonists of the invention, or contiguous nucleotide sequence thereof, do not comprise more than 2 contiguous DNA nucleosides.

XVI. Mixmers and Totalmers

For splice modulation it is often advantageous to use oligonucleotides which do not recruit RNAaseH. As RNaseH activity requires a contiguous sequence of DNA nucleotides, RNaseH activity of oligonucleotides may be achieved by designing oligonucleotides which do not comprise a region of more than 3 or more than 4 contiguous DNA nucleosides. This may be achieved by using oligonucleotides or contiguous nucleoside regions thereof with a mixmer design, which comprise sugar modified nucleosides, such as 2′ sugar modified nucleosides, and short regions of DNA nucleosides, such as 1, 2 or 3 DNA nucleosides. Mixmers are exemplified herein by every second design, wherein the nucleosides alternate between 1 LNA and 1 DNA nucleoside, e.g. LDLDLDLDLDLDLDLL, with 5′ and 3′ terminal LNA nucleosides, and every third design, such as LDDLDDLDDLDDLDDL, where every third nucleoside is a LNA nucleoside.

A totalmer is an oligonucleotide or a contiguous nucleotide sequence thereof which does not comprise DNA or RNA nucleosides, and may for example comprise only 2′-O-MOE nucleosides, such as a fully MOE phosphorothioate, e.g. MMMMMMMMMMMMMMMMMMMM, where M=2′-O-MOE, or may for example comprise only 2′oMe nucleosides, which are reported to be effective splice modulators for therapeutic use.

Alternatively, a mixmer may comprise a mixture of modified nucleosides, such as MLMLMLMLMLMLMLMLMLML, wherein L=LNA and M=2′-O-MOE nucleosides.

Advantageously, the internucleoside nucleosides in mixmers and totalmers may be phosphorothioate, or a majority of nucleoside linkages in mixmers may be phosphorothioate. Mixmers and totalmers may comprise other internucleoside linkages, such as phosphodiester or phosphorodithioate, by way of example.

In some embodiments, the oligonucleotide progranulin agonists are or comprise an oligonucleotide mixmer or totalmer. In some embodiments, the contiguous nucleotide sequence is a mixmer or a totalmer.

XVII. Target Sequence

The oligonucleotide progranulin agonists of the invention target the promoter of the human progranulin gene.

The target sequence may also be referred to as a target nucleic acid or target site sequence.

The term “gene” as used herein, particularly with reference to the progranulin gene or granulin precursor gene (GRN), encompasses both protein coding and non-protein coding sequences. It is understood that such sequences include transcribed and untranscribed sequences, and translated and untranslated sequences. Non-protein coding sequences may comprise regulatory sequences such as enhancers, silencers, promoters, and/or 3′ and 5′ untranslated regions (UTR). The oligonucleotide progranulin agonists of the invention target a promoter region of the progranulin gene.

The term “progranulin nucleic acid sequence” as used herein may also refer to nucleic acid sequences of the progranulin gene in the sense of the definition outlined herein. The target nucleic acid sequences of the progranulin gene may refer to sequences as present within the genomic DNA or the same or antisense sequences present in a cell in any other form, such as mRNA, or other single or double stranded RNAs, such as miRNAs or siRNAs.

Reference to genes and their corresponding nucleotide sequences as used herein is not intended to be necessarily limited to either one of the sense or antisense strands thereof. Accordingly, both the sense and antisense sequences may be encompassed.

The terms progranulin gene and granulin precursor gene are used interchangeably herein.

In some embodiments the human progranulin gene has the sequence of NCBI Reference sequence NG_007886.1.

In some embodiments the promoter of the human progranulin gene comprises a sequence defined by SEQ ID NO: 76. SEQ ID NO: 76 is provided herein as a reference sequence and it will be understood that the target nucleic acid may be an allelic variant of SEQ ID NO: 76, such as an allelic variant, which comprises one or more polymorphisms in the human progranulin nucleic acid sequence.

In some embodiments the promoter of the human progranulin gene consists of SEQ ID NO: 76.

In some embodiments the promoter of the human progranulin gene comprises the intergenic region from SLC25A39 to GRN transcription start codon (SEQ ID NO: 74). SEQ ID NO: 74 is provided herein as a reference sequence and it will be understood that the target nucleic acid may be an allelic variant of SEQ ID NO: 74, such as an allelic variant, which comprises one or more polymorphisms in the human progranulin nucleic acid sequence.

In some embodiments the promoter of the human progranulin gene consists of SEQ ID NO: 74.

In some embodiments, the oligonucleotide progranulin agonist of the invention targets the promoter of the human progranulin gene within position 2757-5357 of NCBI Reference sequence NG_007886.1 (SEQ ID NO: 75). Put another way, in some embodiments, the oligonucleotide progranulin agonist of the invention targets the promoter of the human progranulin gene within position 18009-20609 of SEQ ID NO: 76 (SEQ ID NO: 75).

In some embodiments the promoter of the human progranulin gene consists of SEQ ID NO: 75.

In some embodiments the promoter of the human progranulin gene consists of SEQ ID NO: 75.

In some embodiments, the oligonucleotide progranulin agonist of the invention targets the promoter of the human progranulin gene within position 4007-5213 of NCBI Reference sequence NG_007886.1 (SEQ ID NO: 1).

In some embodiments the promoter of the human progranulin gene comprises SEQ ID NO: 1.

In some embodiments the promoter of the human progranulin gene consists of SEQ ID NO: 1.

In some embodiments the target sequence is a contiguous nucleotide sequence within SEQ ID NO: 1.

SEQ ID NO: 1 is provided herein as a reference sequence and it will be understood that the target progranulin nucleic acid may be an allelic variant of SEQ ID NO: 1, such as an allelic variant which comprises one or more polymorphisms in the human progranulin nucleic acid sequence.

In other embodiments the promoter of the human progranulin gene comprises nucleotides −2423 bp to +207 bp relative to the transcriptional start site as defined in Banzhaf-Strathmann et al., Acta Neuropathologica Communications, 2013, 1(16). This sequence is shown as SEQ ID NO: 77.

In some embodiments the promoter of the human progranulin gene consists of SEQ ID NO: 77.

In some embodiments the promoter of the human progranulin gene consists of SEQ ID NO: 77.

In some embodiments the promoter of the human progranulin gene comprises nucleotides −1065 bp to +135 bp relative to the transcriptional start site as defined in Banzhaf-Strathmann et al., Acta Neuropathologica Communications, 2013, 1(16). This sequence is shown as SEQ ID NO: 78.

In some embodiments the promoter of the human progranulin gene consists of SEQ ID NO: 78.

In some embodiments the promoter of the human progranulin gene consists of SEQ ID NO: 78.

In some embodiments, the contiguous nucleotide sequence of the oligonucleotide progranulin agonist of the invention is complementary, such as fully complementary, to a sequence selected from the group consisting of positions 4137 to 4157, 4237 to 4257, 4574 to 4594, 4789 to 4809, 4015 to 4036, 4035 to 4055, 4056 to 4076, 4113 to 4133, 4169 to 41893, 4205 to 4225, 4278 to 4298, 4302 to 4322, 4409 to 4429, 4487 to 4507, 4526 to 4546, 4603 to 4623, 4691 to 4711, 4815 to 4835, 4850 to 4870, 4882 to 4902, 4911 to 4931, 4971 to 4991, or 5010 to 5030 of NCBI Reference sequence NG_007886.1.

Unless otherwise stated, all ranges are inclusive of the start and end value, e.g. the contiguous nucleotide sequence corresponding to positions 4137 to 4157 comprises 21 nucleotides.

In some embodiments, the contiguous nucleotide sequence of the oligonucleotide progranulin agonist of the invention is complementary, such as fully complementary, to a sequence selected from the group consisting of nucleotides 131 to 151, 231 to 251, 568 to 588, 783 to 803, 9 to 30, 29 to 49, 50 to 70,107 to 127, 163 to 183, 199 to 219, 272 to 292, 296 to 316, 403 to 423, 481 to 501, 520 to 540, 597 to 617, 685 to 705, 809 to 829, 844 to 864, 876 to 896, 905 to 925, 965 to 985, or 1004 to 1024, of SEQ ID NO: 1 or a fragment thereof.

In some embodiments the contiguous nucleotide sequence is complementary, such as fully complementary, to a sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, and SEQ ID NO: 25, or a fragment thereof.

In some embodiments the contiguous nucleotide sequence is complementary, such as fully complementary, to a sequence selected from the group consisting of SEQ ID NO: 2, SEQ ID NO: 4, SEQ ID NO: 9, SEQ ID NO: 10, SEQ ID NO: 11, SEQ ID NO: 12, SEQ ID NO: 15, SEQ ID NO: 17, SEQ ID NO: 18, SEQ ID NO: 20, SEQ ID NO: 21, SEQ ID NO: 23 and SEQ ID NO: 25, or a fragment thereof.

In some embodiments the contiguous nucleotide sequence is complementary, such as fully complementary, to a sequence selected from the group consisting of SEQ ID NO: 9, SEQ ID NO: 11, SEQ ID NO: 17, and SEQ ID NO: 18, or a fragment thereof.

In some embodiments the contiguous nucleotide sequence is complementary, such as fully complementary, to SEQ ID NO: 9, or a fragment thereof.

In some embodiments the contiguous nucleotide sequence is complementary, such as fully complementary, to SEQ ID NO: 11, or a fragment thereof.

In some embodiments the contiguous nucleotide sequence is complementary, such as fully complementary, to SEQ ID NO: 17, or a fragment thereof.

In some embodiments the contiguous nucleotide sequence is complementary, such as fully complementary, to SEQ ID NO: 18, or a fragment thereof.

A fragment of the target sequence may be 8, 9, 10, 11, 12, 13, 14, 15, 16, 17, 18, 19 or 20 nucleotides in length.

In some embodiments the contiguous nucleotide sequence is complementary, such as fully complementary, to at least 8 contiguous nucleotides of any of the target sequences recited herein.

In some embodiments the contiguous nucleotide sequence is complementary, such as fully complementary, to at least 10 contiguous nucleotides of any of the target sequences recited herein.

In some embodiments the contiguous nucleotide sequence is complementary, such as fully complementary, to at least 12 contiguous nucleotides of any of the target sequences recited herein.

In some embodiments the contiguous nucleotide sequence is complementary, such as fully complementary, to at least 14 contiguous nucleotides of any of the target sequences recited herein.

In some embodiments the contiguous nucleotide sequence is complementary, such as fully complementary, to at least 16 contiguous nucleotides of any of the target sequences recited herein.

In some embodiments the contiguous nucleotide sequence is complementary, such as fully complementary, to at least 18 contiguous nucleotides of any of the target sequences recited herein.

In some embodiments the contiguous nucleotide sequence is complementary, such as fully complementary, to at least 20 contiguous nucleotides of any of the target sequences recited herein.

XVIII. Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U).

It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pairs) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

In certain embodiments of the invention the oligonucleotide progranulin agonist is a double stranded oligonucleotide, such as an saRNA. In these embodiments the double stranded oligonucleotide may have a nucleotide overhang, such as a 2 nucleotide overhang which may be at the 3′ end of the contiguous nucleotide sequence. In such embodiments complementarity is defined based upon the double stranded sequence without the overhang. For example, if the oligonucleotide is 21 nucleotides in length and includes a 2 nucleotide overhang, complementarity is determined based upon the 19 nucleotides without the two nucleotide overhang.

Within the present invention, the term “complementary” requires the contiguous nucleotide sequence to be at least about 80% complementary, or at least about 90% complementary, to the target sequence, i.e. the promoter of the human progranulin gene. In some embodiments the oligonucleotide progranulin agonist may be at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% complementary the target sequence, i.e. the promoter of the human progranulin gene. Put another way, in some embodiments, the contiguous nucleotide sequence within an oligonucleotide progranulin agonist of the invention may include one, two, three or more mis-matches, wherein a mis-match is a nucleotide within the contiguous nucleotide sequence which does not base pair with its target.

The term “fully complementary”, refers to 100% complementarity.

In some embodiments the contiguous nucleotide sequence is fully complementary to the target sequence.

XIX. Identity

The term “identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif).

The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

It is therefore to be understood that there is a relationship between identity and complementarity such that a contiguous nucleotide sequence within an oligonucleotide progranulin agonist of the invention that is complementary to a target sequence also shares a percentage of identity with said complementary sequence.

XX. Hybridization

The terms “hybridizing” or “hybridizes” as used herein are to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTln(K_{d}), where R is the gas constant and T is the absolute temperature. Therefore, a very low AG° of the reaction between an oligonucleotide and the target nucleic acid reflects a strong hybridization between the oligonucleotide and target nucleic acid. AG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405.

In some embodiments, oligonucleotide progranulin agonists of the present invention hybridize to a target nucleic acid with estimated ΔG° values below −10 kcal for oligonucleotides that are 10-30 nucleotides in length.

In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal, or −16 to −27 kcal such as −18 to −25 kcal.

XXI. Double Stranded Oligonucleotide Progranulin Agonists

In some embodiments, the oligonucleotide progranulin agonist is a double stranded oligonucleotide.

In some embodiments the double stranded oligonucleotide progranulin agonist is a short activating RNA (saRNAs).

The term “short activating RNA” (saRNA), as used herein, refers to small double stranded RNA that is typically 21 nucleotides in length and may comprise a 2 nucleotide overhang at the 3′ end. saRNAs are capable of inducing gene activation by a process known as RNA activation (RNAa), wherein gene activation is induced by hybridization of the saRNA with a target nucleic acid sequence. Said target nucleic acid sequences typically comprise promoter regions of a gene.

A known mechanism of transcriptional upregulation by saRNAs involves Ago2, and is associated with epigenetic modification at target sites, e.g. promotors. Argo2 associates with an saRNA, which guides the complex to a target and facilitates the assembly of an RNA-induced transcriptional activation (RITA) complex. RITA-RNA polymerase II interactions are thought to promote transcription initiation and productive elongation, as well as monoubiquitination of histone 2B (Portnoy et al., Cell Res., 2016, 26(3), 320-335).

In some embodiments, the oligonucleotide progranulin agonist of the invention, such as an saRNA, is 19 nucleotides in length.

In some embodiments, the oligonucleotide progranulin agonist of the invention, such as an saRNA is 20 nucleotides in length.

In some embodiments, the oligonucleotide progranulin agonist of the invention, such as an saRNA, is 21 nucleotides in length.

In some embodiments, the oligonucleotide progranulin agonist of the invention, such as an saRNA, is 22 nucleotides in length.

It will be understood that when discussing embodiments including a double stranded oligonucleotide the length measurement refers to the length of one of the strands. In embodiments where the two strands may not be the same length, the length is taken as the length of the longest strand.

In some embodiments, the oligonucleotide progranulin agonist of the invention, such as an saRNA, may have a nucleotide overhang. The nucleotide overhang may be a 2 nucleotide overhang. In some embodiments the overhang may be at the 3′ end of the contiguous nucleotide sequence. In some embodiments the overhang may comprise or consist of two thymine nucleotides (TT).

In some embodiments, the contiguous nucleotide sequence is selected from the group consisting of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 79, and SEQ ID NO: 82, or at least 8 contiguous nucleotides thereof.

It will be apparent to the skilled person that a double stranded oligonucleotide can be defined by reference to either the sense strand or the antisense strand.

In some embodiments, the sense strand of the contiguous nucleotide sequence is a sequence selected from the group consisting of SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 79, or at least 10 contiguous nucleotides thereof.

In some embodiments, the sense strand of the contiguous nucleotide sequence is a sequence selected from the group consisting of SEQ ID NO: 26, SEQ ID NO: 30, SEQ ID NO: 40, SEQ ID NO: 42, SEQ ID NO: 44, SEQ ID NO: 46, SEQ ID NO: 52, SEQ ID NO: 56, SEQ ID NO: 58, SEQ ID NO: 62, SEQ ID NO: 64, SEQ ID NO: 68, SEQ ID NO: 72, and SEQ ID NO: 79, or at least 10 contiguous nucleotides thereof.

In some embodiments, the sense strand of the contiguous nucleotide sequence is a sequence selected from the group consisting of SEQ ID NO: 40, SEQ ID NO: 44, SEQ ID NO: 56, SEQ ID NO: 58, and SEQ ID NO: 79, or at least 10 contiguous nucleotides thereof.

In some embodiments, the sense strand of the contiguous nucleotide sequence is SEQ ID NO: 40, or at least 10 contiguous nucleotides thereof.

In some embodiments, the sense strand of the contiguous nucleotide sequence is SEQ ID NO: 44, or at least 10 contiguous nucleotides thereof.

In some embodiments, the sense strand of the contiguous nucleotide sequence is SEQ ID NO: 56, or at least 10 contiguous nucleotides thereof.

In some embodiments, the sense strand of the contiguous nucleotide sequence is SEQ ID NO: 58, or at least 10 contiguous nucleotides thereof.

In some embodiments, the sense strand of the contiguous nucleotide sequence is SEQ ID NO: 79, or at least 10 contiguous nucleotides thereof.

In some embodiments, the antisense strand of the contiguous nucleotide sequence is a sequence selected from the group consisting of SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, and SEQ ID NO: 82, or at least 10 contiguous nucleotides thereof.

In some embodiments, the antisense strand of the contiguous nucleotide sequence is a sequence selected from the group consisting of SEQ ID NO: 27, SEQ ID NO: 31, SEQ ID NO: 41, SEQ ID NO: 43, SEQ ID NO: 45, SEQ ID NO: 47, SEQ ID NO: 53, SEQ ID NO: 57, SEQ ID NO: 59, SEQ ID NO: 63, SEQ ID NO: 65, SEQ ID NO: 69, SEQ ID NO: 73, and SEQ ID NO: 82, or at least 10 contiguous nucleotides thereof.

In some embodiments, the antisense strand of the contiguous nucleotide sequence is a sequence selected from the group consisting of SEQ ID NO: 41, SEQ ID NO: 45, SEQ ID NO: 57, SEQ ID NO: 59, and SEQ ID NO: 82, or at least 10 contiguous nucleotides thereof.

In some embodiments, the antisense strand of the contiguous nucleotide sequence is SEQ ID NO: 41, or at least 10 contiguous nucleotides thereof.

In some embodiments, the antisense strand of the contiguous nucleotide sequence is SEQ ID NO: 45, or at least 10 contiguous nucleotides thereof.

In some embodiments, the antisense strand of the contiguous nucleotide sequence is SEQ ID NO: 57, or at least 10 contiguous nucleotides thereof.

In some embodiments, the antisense strand of the contiguous nucleotide sequence is SEQ ID NO: 59, or at least 10 contiguous nucleotides thereof.

In some embodiments, the antisense strand of the contiguous nucleotide sequence is SEQ ID NO: 82, or at least 10 contiguous nucleotides thereof.

In certain embodiments wherein the oligonucleotide is a double stranded oligonucleotide the contiguous nucleotide sequence may be a fragment of 10, 11, 12, 13, 14, 15, 16, 17, 18, 19 or 20 nucleotides of any of the sequences recited herein.

It will be understood that double stranded oligonucleotides, such as saRNAs, comprise two complementary strands, each of which may hybridize with complementary oligonucleotide sequences, such as oligonucleotide sequences of endogenous RNAs or DNA.

Without wishing to be bound by theory, it will be appreciated that a double stranded oligonucleotide of the invention may act through binding to the sense strand, the antisense strand or both strands of the target sequence.

XXII. Single Stranded Oligonucleotide Progranulin Agonists

In some embodiments, the oligonucleotide progranulin agonist is a single stranded oligonucleotide.

In some embodiments the single stranded oligonucleotide progranulin agonist is an antisense oligonucleotide.

The term “antisense oligonucleotide” as used herein is defined as an oligonucleotide capable of modulating expression of a target gene by hybridizing to a target nucleic acid, in particular to a contiguous sequence on a target nucleic acid. Antisense oligonucleotides are not essentially double stranded and are therefore not siRNAs or shRNAs. The oligonucleotides of the present invention may be single stranded. It is understood that single stranded oligonucleotides of the present invention can form hairpins or intermolecular duplex structures (duplex between two molecules of the same oligonucleotide), as long as the degree of intra or inter self-complementarity is less than approximately 50% across of the full length of the oligonucleotide.

In some embodiments, the single stranded antisense oligonucleotide of the invention may not contain RNA nucleosides.

Advantageously, the antisense oligonucleotide of the invention comprises one or more modified nucleosides or nucleotides, such as 2′ sugar modified nucleosides. Furthermore, in some antisense oligonucleotides of the invention, it may be advantageous that the nucleosides which are not modified are DNA nucleosides.

It will be apparent to the skilled person that a single stranded oligonucleotide progranulin agonist of the invention may include a contiguous nucleotide sequence which is complementary to the sense strand or the antisense strand of the target sequence. The present invention contemplates both of these embodiments.

In some embodiments, the single stranded oligonucleotide progranulin agonist of the invention comprises a contiguous nucleotide sequence that is complementary to the sense strand of the human progranulin gene, NCBI Reference Sequence: NG_007886.1.

In some embodiments, the oligonucleotide progranulin agonist of the invention comprises a contiguous nucleotide sequence that is complementary to the antisense strand of the human progranulin gene, NCBI Reference Sequence: NG_007886.1.

In some embodiments, the oligonucleotide progranulin agonist of the invention comprises a contiguous nucleotide sequence that is fully complementary to a sequence of either the sense or antisense strand of the human progranulin gene, NCBI Reference Sequence: NG_007886.1.

In some embodiments, the oligonucleotide progranulin agonist of the invention comprises a contiguous nucleotide sequence that is complementary to a portion of the sense strand of SEQ ID NO: 1.

In some embodiments, the oligonucleotide progranulin agonist of the invention comprises a contiguous nucleotide sequence that is complementary to a portion of the antisense strand of SEQ ID NO: 1.

The invention further provides for oligonucleotide progranulin agonists, wherein the oligonucleotide is single stranded, and wherein the contiguous nucleotide sequence is complementary to the sense strand of the human progranulin gene.

In some embodiments, the contiguous nucleotide sequence is selected from the group consisting of SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, and SEQ ID NO: 82, or at least 10 contiguous nucleotides thereof.

In some embodiments, the contiguous nucleotide sequence is selected from the group consisting of SEQ ID NO: 27, SEQ ID NO: 31, SEQ ID NO: 41, SEQ ID NO: 43, SEQ ID NO: 45, SEQ ID NO: 47, SEQ ID NO: 53, SEQ ID NO: 57, SEQ ID NO: 59, SEQ ID NO: 63, SEQ ID NO: 65, SEQ ID NO: 69, SEQ ID NO: 73, and SEQ ID NO: 82, or at least 10 contiguous nucleotides thereof.

In some embodiments, the contiguous nucleotide sequence is a sequence selected from the group consisting of SEQ ID NO: 41, SEQ ID NO: 45, SEQ ID NO: 57, SEQ ID NO: 59, and SEQ ID NO: 82, or at least 10 contiguous nucleotides thereof.

In some embodiments, the contiguous nucleotide sequence is SEQ ID NO: 41, or at least 10 contiguous nucleotides thereof.

In some embodiments, the contiguous nucleotide sequence is SEQ ID NO: 45, or at least 10 contiguous nucleotides thereof.

In some embodiments, the contiguous nucleotide sequence is SEQ ID NO: 57, or at least 10 contiguous nucleotides thereof.

In some embodiments, the contiguous nucleotide sequence is SEQ ID NO: 59, or at least 10 contiguous nucleotides thereof.

In some embodiments, the contiguous nucleotide sequence is SEQ ID NO: 82, or at least 10 contiguous nucleotides thereof.

The invention further provides for oligonucleotide progranulin agonists, wherein the oligonucleotide is single stranded, and wherein the contiguous nucleotide sequence is complementary to the antisense strand of the human progranulin gene.

In some embodiments, the contiguous nucleotide sequence is selected from the group consisting of SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, and SEQ ID NO: 79, or at least 10 contiguous nucleotides thereof.

In some embodiments, the contiguous nucleotide sequence is selected from the group consisting of SEQ ID NO: 26, SEQ ID NO: 30, SEQ ID NO: 40, SEQ ID NO: 42, SEQ ID NO: 44, SEQ ID NO: 46, SEQ ID NO: 52, SEQ ID NO: 56, SEQ ID NO: 58, SEQ ID NO: 62, SEQ ID NO: 64, SEQ ID NO: 68, SEQ ID NO: 72, and SEQ ID NO: 79, or at least 10 contiguous nucleotides thereof.

In some embodiments, the contiguous nucleotide sequence is selected from the group consisting of SEQ ID NO: 40, SEQ ID NO: 44, SEQ ID NO: 56, SEQ ID NO: 58, and SEQ ID NO: 79, or at least 10 contiguous nucleotides thereof.

In some embodiments, the contiguous nucleotide sequence is SEQ ID NO: 40, or at least 10 contiguous nucleotides thereof.

In some embodiments, the contiguous nucleotide sequence is SEQ ID NO: 44, or at least 10 contiguous nucleotides thereof.

In some embodiments, the contiguous nucleotide sequence is SEQ ID NO: 56, or at least 10 contiguous nucleotides thereof.

In some embodiments, the contiguous nucleotide sequence is SEQ ID NO: 58, or at least 10 contiguous nucleotides thereof.

In some embodiments, the contiguous nucleotide sequence is SEQ ID NO: 79, or at least 10 contiguous nucleotides thereof.

In certain embodiments wherein the oligonucleotide is a single stranded oligonucleotide, the contiguous nucleotide sequence may be a fragment of 10, 11, 12, 13, 14, 15, 16, 17, 18, 19 or 20 nucleotides of any of the sequences recited herein.

XXIII. Region D′ or D″ in an Oligonucleotide

The oligonucleotide progranulin agonist of the invention may in some embodiments comprise or consist of the contiguous nucleotide sequence of the oligonucleotide which is complementary to the target nucleic acid, such as a mixmer or totalmer region, and further 5′ and/or 3′ nucleosides. The further 5′ and/or 3′ nucleosides may or may not be complementary, such as fully complementary, to the target nucleic acid. Such further 5′ and/or 3′ nucleosides may be referred to as region D′ and D″ herein.

The addition of region D′ or D″ may be used for the purpose of joining the contiguous nucleotide sequence, such as the mixmer or totalmer, to a conjugate moiety or another functional group. When used for joining the contiguous nucleotide sequence with a conjugate moiety is can serve as a biocleavable linker. Alternatively, it may be used to provide exonucleoase protection or for ease of synthesis or manufacture.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs within a single oligonucleotide.

In one embodiment the oligonucleotide progranulin agonist of the invention comprises a region D′ and/or D″ in addition to the contiguous nucleotide sequence which constitutes a mixmer or a totalmer.

In some embodiments the intemucleoside linkage positioned between region D′ or D″ and the mixmer or totalmer region is a phosphodiester linkage.

XXIV. Conjugate

The invention encompasses an oligonucleotide progranulin agonist covalently attached to at least one conjugate moiety. In some embodiments this may be referred to as a conjugate of the invention.

In some embodiments, the invention provides oligonucleotide progranulin agonists covalently attached to at least one conjugate moiety.

The term “conjugate” as used herein refers to an oligonucleotide progranulin agonist which is covalently linked to a non-nucleotide moiety (conjugate moiety or region C or third region). The conjugate moiety may be covalently linked to the oligonucleotide, optionally via a linker group, such as region D′ or D″.

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103.

In some embodiments, the non-nucleotide moiety (conjugate moiety) is selected from the group consisting of carbohydrates (e.g. GalNAc), cell surface receptor ligands, drug substances, hormones, lipophilic substances, polymers, proteins, peptides, toxins (e.g. bacterial toxins), vitamins, viral proteins (e.g. capsids) or combinations thereof.

XXV. Linkers

A linkage or linker is a connection between two atoms that links one chemical group or segment of interest to another chemical group or segment of interest via one or more covalent bonds. Conjugate moieties can be attached to the oligonucleotide progranulin agonist directly or through a linking moiety (e.g. linker or tether). Linkers serve to covalently connect a third region, e.g. a conjugate moiety (Region C), to a first region, e.g. an oligonucleotide or contiguous nucleotide sequence complementary to the target nucleic acid (region A).

In some embodiments of the invention the conjugate or oligonucleotide progranulin agonist conjugate of the invention may optionally comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In some embodiments the nuclease susceptible linker comprises between 1 and 5 nucleosides, such as DNA nucleoside(s) comprising at least two consecutive phosphodiester linkages. Phosphodiester containing biocleavable linkers are described in more detail in WO 2014/076195.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups. The oligonucleotide progranulin agonist conjugates of the present invention can be constructed of the following regional elements A-C, A-B-C, A-B-Y-C, A-Y-B-C or A-Y-C. In some embodiments the linker (region Y) is an amino alkyl, such as a C2 — C36 amino alkyl group, including, for example C6 to C12 amino alkyl groups. In some embodiments the linker (region Y) is a C6 amino alkyl group.

XXVI. Salts

The term “salts” as used herein conforms to its generally known meaning, i.e. an ionic assembly of anions and cations.

The invention provides for pharmaceutically acceptable salts of the oligonucleotide progranulin agonists according to the invention, or the conjugate according to the invention.

The invention provides for oligonucleotide progranulin agonists according to the invention wherein the oligonucleotide progranulin agonists are in the form of a pharmaceutically acceptable salt. In some embodiments the pharmaceutically acceptable salt may be a sodium salt or a potassium salt.

The invention provides for a pharmaceutically acceptable sodium salt of the oligonucleotide progranulin agonist according to the invention, or the conjugate according to the invention.

The invention provides for a pharmaceutically acceptable potassium salt of the oligonucleotide progranulin agonist according to the invention, or the conjugate according to the invention.

XXVII. Pharmaceutical Compositions

The invention provides for a pharmaceutical composition comprising the oligonucleotide progranulin agonist of the invention, or the conjugate or salt of the invention, and a pharmaceutically acceptable diluent, solvent, carrier, salt and/or adjuvant.

The invention provides for a pharmaceutical composition comprising the oligonucleotide progranulin agonists of the invention, or the conjugate of the invention, and a pharmaceutically acceptable salt. For example, the salt may comprise a metal cation, such as a sodium salt or a potassium salt.

The invention provides for a pharmaceutical composition according to the invention, wherein the pharmaceutical composition comprises the oligonucleotide progranulin agonist of the invention or the conjugate of the invention, or the pharmaceutically acceptable salt of the invention, and an aqueous diluent or solvent.

The invention provides for a solution, such as a phosphate buffered saline solution of the oligonucleotide progranulin agonist of the invention, or the conjugate of the invention, or the pharmaceutically acceptable salt of the invention. Suitably the solution, such as phosphate buffered saline solution, of the invention is a sterile solution.

XXVIII. Method for Regulating Progranulin Expression

The invention provides for a method for enhancing, upregulating or restoring the expression of progranulin in a cell, such as a cell which is expressing progranulin, said method comprising administering an oligonucleotide progranulin agonist of the invention or a conjugate of the invention, or a salt of the invention, or the pharmaceutical composition of the invention in an effective amount to said cell.

In some embodiments the method is an in vitro method.

In some embodiments the method is an in vivo method.

In some embodiments, the cell is either a human cell or a mammalian cell.

In some embodiments, the cell is part of, or derived from, a subject suffering from or susceptible to a disease associated with progranulin or progranulin haploinsufficiency, such as a neurological disease. Such neurological diseases include but are not limited to frontotemporal dementia (FTD), amyotrophic lateral sclerosis (ALS), frontotemporal dementia with neuropathologic frontotemporal lobar degeneration (FTLD), familial frontotemporal dementia with neuropathologic frontotemporal lobar degeneration associated with accumulation of TDP-43 inclusions (FTLD-TDP), and neuronal ceroid lipofuscinosis (NCL).

Treatment

The invention provides for a method for treating or preventing neurological disease, comprising administering a therapeutically or prophylactically effective amount of an oligonucleotide progranulin agonist of the invention, or a conjugate of the invention, or a salt of the invention, or a pharmaceutical composition of the invention to a subject suffering from or susceptible to neurological disease. In one embodiment the neurological disease may be a TDP-43 pathology.

The invention provides for a method for treating progranulin haploinsufficiency, comprising administering a therapeutically or prophylactically effective amount of an oligonucleotide progranulin agonist of the invention or a conjugate of the invention, or a salt of the invention, or a pharmaceutical composition of the invention to a subject suffering from progranulin haploinsufficiency or a related disorder.

In some embodiments, the subject is an animal, preferably a mammal such as a mouse, rat, hamster, or monkey, or preferably a human.

The invention provides for an oligonucleotide progranulin agonist of the invention for use as a medicament.

The invention provides for an oligonucleotide progranulin agonist of the invention for use in therapy.

The invention provides for a oligonucleotide progranulin agonist of the invention, or a conjugate of the invention, or a salt of the invention, or a pharmaceutical composition of the invention, for use as a medicament.

The invention provides an oligonucleotide progranulin agonist of the invention, or a conjugate of the invention, or a salt according to the invention, or a pharmaceutical composition according to the invention for use in therapy.

The invention provides for an oligonucleotide progranulin agonist of the invention, or a conjugate of the invention, or a salt of the invention, or a pharmaceutical composition of the invention for use in the treatment of a neurological disease. In one embodiment the neurological disease may be a TDP-43 pathology.

The invention provides for an oligonucleotide progranulin agonist of the invention, or a conjugate of the invention, or a salt of the invention, or a pharmaceutical composition of the invention for use in the treatment of progranulin haploinsufficiency, or a related disorder.

The invention provides for the use of an oligonucleotide progranulin agonist of the invention or a conjugate of the invention, or a salt of the invention, or a pharmaceutical composition of the invention, for the preparation of a medicament for the treatment or prevention of a neurological disease. In one embodiment the neurological disease may be a TDP-43 pathology.

The invention provides for the use of the oligonucleotide progranulin agonist of the invention, or a conjugate of the invention, or a salt of the invention, or a pharmaceutical composition of the invention, for the preparation of a medicament for the treatment of progranulin haploinsufficiency or a related disorder.

In some embodiments composition for use, the method or the use of the invention is for the treatment of frontotemporal dementia (FTD), neuropathologic frontotemporal lobar degeneration or neuroinflammation. In other embodiments the composition for use, method or use of the invention is for the treatment of amyotrophic lateral sclerosis (ALS), Alzheimer's disease, Parkinson's disease, Autism, Hippocampal sclerosis dementia, Down syndrome, Huntington's disease, polyglutamine diseases, spinocerebellar ataxia 3, myopathies or Chronic Traumatic Encephalopathy.

XXIX. TDP-43 Pathologies

A TDP-43 pathology is a disease which is associated with reduced or aberrant expression of TDP-43, often associated with an increase in cytoplasmic TDP-43, particularly hyper-phosphorylated and ubiquitinated TDP-43.

Diseases associated with TDP-43 pathology include amyotrophic lateral sclerosis (ALS), frontotemporal lobar degeneration (FTLD), Alzheimer's disease, Parkinson's disease, Autism, Hippocampal sclerosis dementia, Down syndrome, Huntington's disease, polyglutamine diseases, such as spinocerebellar ataxia 3, myopathies and Chronic Traumatic Encephalopathy.

XXX. Progranulin Upregulation

In certain embodiments the oligonucleotide progranulin agonists of the present invention may enhance the production of their progranulin mRNA by at least about 10%. In other embodiments the oligonucleotide progranulin agonist of the present invention may enhance the production of progranulin mRNA by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, or at least about 600% or more.

In certain embodiments the oligonucleotide progranulin agonists of the present invention may enhance the production of their progranulin protein by at least about 10%. In other embodiments the oligonucleotide progranulin agonist of the present invention may enhance the production of progranulin protein by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, or at least about 600% or more.

TABLE 1
saRNA sequences of the invention and their target sequences

| | Target | | | | Sense strand | | Antisense strand | |
|---|---|---|---|---|---|---|---|---|
| | | SEQ | | | | SEQ | | SEQ |
| | | ID | Position in | Position in | | ID | | ID |
| saRNA | Sequence | NO: | NG_007886.1 | SEQ ID NO: 1 | Sequence (5′-3′) | NO: | Sequence (5′-3′) | NO: |
| SARNA1 | AAATTAGATTC | 2 | 4137 to 4157 | 131 to 151 | (AUU AGA UUC AGC CUG | 26 | (GUC UCA GGC UGA AUC | 27 |
| | AGCCTGAGAC | | | | AGA C)TT | | UAA U)TT | |
| SARNA2 | AAACCATCCTT | 3 | 4237 to 4257 | 231 to 251 | (ACC AUC CUU CAA CUC | 28 | (GAG UGA GUU GAA GGA | 29 |
| | CAACTCACTC | | | | ACU C)TT | | UGG U)TT | |
| SARNA3 | AATCGAGACCA | 4 | 4574 to 4594 | 568 to 588 | (UCG AGA CCA UCC UGG | 30 | (UUG GCC AGG AUG GUC | 31 |
| | TCCTGGCCAA | | | | CCA A)TT | | UCG A)TT | |
| SARNA4 | AACAAAAGGAT | 5 | 4789 to 4809 | 783 to 803 | (CAA AAG GAU AGA AAG | 32 | (UCG CCU UUC UAU CCU | 33 |
| | AGAAAGGCGA | | | | GCG A)TT | | UUU G)TT | |
| SARNA5 | AATCTTCCCAG | 6 | 4015 to 4036 | 9 to 30 | (UCU UCC CAG ACU CAG | 34 | (UUG AGC UGA GUC UGG | 35 |
| | ACTCAGCTCAA | | | | CUC AA)TT | | GAA GA)TT | |
| SARNA6 | AAGGAGATGCT | 7 | 4035 to 4055 | 29 to 49 | (GGA GAU GCU CCU AAG | 36 | (CCA CCU UAG GAG CAU | 37 |
| | CCTAAGGTGG | | | | GUG G)TT | | CUC C)TT | |
| SARNA7 | AATGAAATCTC | 8 | 4056 to 4076 | 50 to 70 | (UGA AAU CUC UUC UUC | 38 | (UGG GGA AGA AGA GAU | 39 |
| | TTCTTCCCCA | | | | CCC A)TT | | UUC A)TT | |
| SARNA8 | AACTGGCGCAC | 9 | 4113 to 4133 | 107 to 127 | (CUG GCG CAC AAC CUU | 40 | (AUA CAA GGU UGU GCG | 41 |
| | AACCTTGTAT | | | | GUA U)TT | | CCA G)TT | |
| SARNA9 | AATCCCTGCTC | 10 | 4169 to 4189 | 163 to 183 | (UCC CUG CUC CCU GUC | 42 | (GCA GGA CAG GGA GCA | 43 |
| | CCTGTCCTGC | | | | CUG C)TT | | GGG A)TT | |
| SARNA10 | AAGGGCTTTGG | 11 | 4205 to 4225 | 199 to 219 | (GGG CUU UGG GGC UGU | 44 | (AUA AAC AGC CCC AAA | 45 |
| | GGCTGTTTAT | | | | UUA U)TT | | GCC C)TT | |
| SARNA11 | AACCGAGTTTG | 12 | 4278 to 4298 | 272 to 292 | (CCG AGU UUG GAG AAU | 46 | (CCC UAU UCU CCA AAC | 47 |
| | GAGAATAGGG | | | | AGG G)TT | | UCG G)TT | |
| SARNA12 | AATAGGGCAGG | 13 | 4302 to 4322 | 296 to 316 | (UAG GGC AGG ACA GGA | 48 | (CCU GUC CUG UCC UGC | 49 |
| | ACAGGACAGG | | | | CAG G)TT | | CCU A)TT | |
| SARNA14 | AATCGCTTTGG | 15 | 4409 to 4429 | 403 to 423 | (UCG CUU UGG GAG CAG | 52 | (ACU CCU GCU CCC AAA | 53 |
| | GAGCAGGAGT | | | | GAG U)TT | | GCG A)TT | |
| SARNA15 | AACAAACACAC | 16 | 4487 to 4507 | 481 to 501 | (CAA ACA CAC AAG UCC | 54 | (GCC CGG ACU UGU GUG | 55 |
| | AAGTCCGGGC | | | | GGG C)TT | | UUU G)TT | |
| SARNA16 | AATCTTAGCAC | 17 | 4526 to 4546 | 520 to 540 | (UCU UAG CAC UUU GGG | 56 | (GCC UCC CAA AGU GCU | 57 |
| | TTTGGGAGGC | | | | AGG C)TT | | AAG A)TT | |
| SARNA17 | AACCCCGTCTC | 18 | 4603 to 4623 | 597 to 617 | (CCC CGU CUC UAC UAA | 58 | (AUU UUU AGU AGA GAC | 59 |
| | TACTAAAAAT | | | | AAA U)TT | | GGG G)TT | |
| SARNA18 | AATCGCTTGAA | 19 | 4691 to 4711 | 685 to 705 | (UCG CUU GAA CCC GGG | 60 | (GCC UCC CGG GUU CAA | 61 |
| | CCCGGGAGGC | | | | AGG C)TT | | GCG A)TT | |
| SARNA19 | AATATTCCCAA | 20 | 4815 to 4835 | 809 to 829 | (UAU UCC CAA UUC AUA | 62 | (GUG UUA UGA AUU GGG | 63 |
| | TTCATAACAC | | | | ACA C)TT | | AAU A)TT | |
| SARNA20 | AATGCCCCAGA | 21 | 4850 to 4870 | 844 to 864 | (UGC CCC AGA CAC GCG | 64 | (AUA GCG CGU GUC UGG | 65 |
| | CACGCGCTAT | | | | CUA U)TT | | GGC A)TT | |
| SARNA21 | AACTCCCCCAG | 22 | 4882 to 4902 | 876 to 896 | (CUC CCC CAG GCG CCU | 66 | (CUG CAG GCG CCU GGG | 67 |
| | GCGCCTGCAG | | | | GCA G)TT | | GGA G)TT | |
| SARNA22 | AAGGAAGGCGA | 23 | 4911 to 4931 | 905 to 925 | (GGA AGG CGA CGA GCA | 68 | (CUG GUG CUC GUC GCC | 69 |
| | CGAGCACCAG | | | | CCA G)TT | | UUC C)TT | |
| SARNA23 | AATCACATGAT | 24 | 4971 to 4991 | 965 to 985 | (UCA CAU GAU CCC UAG | 70 | (AUU UCU AGG GAU CAU | 71 |
| | CCCTAGAAAT | | | | AAA U)TT | | GUG A)TT | |
| SARNA24 | AAGCAGGGAGG | 25 | 5010 to 5030 | 1004 to 1024 | (GCA GGG AGG AGA GUG | 72 | (AAA UCA CUC UCC UCC | 73 |
| | AGAGTGATTT | | | | AUU U)TT | | CUG C)TT | |
| SARNA25 | AACTGGCGCAC | 9 | 4113 to 4133 | 107 to 127 | (CGU UCG CAC AAC CUU | 79 | AUA CAA GGU UGU GCG | 82 |
| | AACCTTGTAT | | | | GUA U)TT | | AAC G)TT | |
| | (with seed | | | | | | | |
| | mutation in | | | | | | | |
| | sense and | | | | | | | |
| | antisense | | | | | | | |
| | strand) | | | | | | | |
| SARNA26 | AACTGGCGCAC | 9 | 4113 to 4133 | 107 to 127 | (CUG GCG CAC AAC CUU | 80 | (AGC AAA GGU UGU GCG | 83 |
| | AACCTTGTAT | | | | UGC U)TT | | CCA G)TT | |
| | (with seed | | | | | | | |
| | mutation in | | | | | | | |
| | sense and | | | | | | | |
| | antisense | | | | | | | |
| | strand) | | | | | | | |
| SARNA27 | AACTGGCGCAC | 9 | 4113 to 4133 | 107 to 127 | (CGU UCG CAC AAC CUU | 81 | (AGC AAA GGU UGU GCG | 84 |
| | AACCTTGTAT | | | | UGC U)TT | | AAC G)TT | |
| | (with seed | | | | | | | |
| | mutation in | | | | | | | |
| | sense and | | | | | | | |
| | antisense | | | | | | | |
| | strand) | | | | | | | |

XXXI. Numbered Embodiments of the Invention

1. An oligonucleotide progranulin agonist, wherein the oligonucleotide is 8-40 nucleotides in length and comprises a contiguous sequence of 8-40 nucleotides in length, which is complementary to the promoter of the human progranulin gene.

2. The oligonucleotide progranulin agonist of embodiment 1, wherein the contiguous nucleotide sequence is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

3. The oligonucleotide progranulin agonist of embodiment 1 or embodiment 2, wherein the contiguous nucleotide sequence is at least 12 nucleotides in length.

4. The oligonucleotide progranulin agonist of any one of embodiments 1 to 3, wherein the contiguous nucleotide sequence is 21 nucleotides in length.

5. The oligonucleotide progranulin agonist of any one of embodiments 1 to 4, wherein the oligonucleotide is the same length as the contiguous nucleotide sequence.

6. The oligonucleotide progranulin agonist of any one of embodiments 1 to 5, wherein the promoter of the human progranulin gene comprises SEQ ID NO: 76.

7. The oligonucleotide progranulin agonist of embodiment 6, wherein the promoter of the human progranulin gene consists of SEQ ID NO: 76.

8. The oligonucleotide progranulin agonist of any one of embodiments 1 to 7, wherein the promoter of the human progranulin gene comprises SEQ ID NO: 75.

9. The oligonucleotide progranulin agonist of embodiment 8, wherein the promoter of the human progranulin gene consists of SEQ ID NO: 75.

10. The oligonucleotide progranulin agonist of any one of embodiments 1 to 9, wherein the promoter of the human progranulin gene comprises SEQ ID NO: 1.

11. The oligonucleotide progranulin agonist of embodiment 10, wherein the promoter of the human progranulin gene consists of SEQ ID NO: 1.

12. The oligonucleotide progranulin agonist of any one of embodiments 1 to 7, wherein the promoter of the human progranulin gene comprises SEQ ID NO: 77.

13. The oligonucleotide progranulin agonist of embodiment 12, wherein the promoter of the human progranulin gene consists of SEQ ID NO: 77.

14. The oligonucleotide progranulin agonist of any one of embodiments 1 to 7, wherein the promoter of the human progranulin gene comprises SEQ ID NO: 78.

15. The oligonucleotide progranulin agonist of embodiment 14, wherein the promoter of the human progranulin gene consists of SEQ ID NO: 78.

16. The oligonucleotide progranulin agonist of any one of embodiments 1 to 7, wherein the promoter of the human progranulin gene comprises SEQ ID NO: 74.

17. The oligonucleotide progranulin agonist of embodiment 16, wherein the promoter of the human progranulin gene consists of SEQ ID NO: 74.

18. The oligonucleotide progranulin agonist of any one of embodiments 1 to 17, wherein the contiguous nucleotide sequence is complementary to a sequence selected from the group consisting of nucleotides 131 to 151, 231 to 251, 568 to 588, 783 to 803, 9 to 30, 29 to 49, 50 to 70,107 to 127, 163 to 183, 199 to 219, 272 to 292, 296 to 316, 403 to 423, 481 to 501, 520 to 540, 597 to 617, 685 to 705, 809 to 829, 844 to 864, 876 to 896, 905 to 925, 965 to 985, and 1004 to 1024 of SEQ ID NO: 1, or a fragment thereof.

19. The oligonucleotide progranulin agonist of any one of embodiments 1 to 18, wherein the contiguous nucleotide sequence is complementary to a sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, and SEQ ID NO: 25, or a fragment thereof.

20. The oligonucleotide progranulin agonist of embodiment 19, wherein the contiguous nucleotide sequence is complementary to a sequence selected from the group consisting of SEQ ID NO: 2, SEQ ID NO: 4, SEQ ID NO: 9, SEQ ID NO: 10, SEQ ID NO: 11, SEQ ID NO: 12, SEQ ID NO: 15, SEQ ID NO: 17, SEQ ID NO: 18, SEQ ID NO: 20, SEQ ID NO: 21, SEQ ID NO: 23 and SEQ ID NO: 25, or a fragment thereof.

21. The oligonucleotide progranulin agonist of embodiment 20, wherein the contiguous nucleotide sequence is complementary to a sequence selected from the group consisting of SEQ ID NO: 9, SEQ ID NO: 11, SEQ ID NO: 17, and SEQ ID NO: 18, or a fragment thereof.

22. The oligonucleotide progranulin agonist of embodiment 21, wherein the contiguous nucleotide sequence is complementary to SEQ ID NO: 9, or a fragment thereof.

23. The oligonucleotide progranulin agonist of embodiment 21, wherein the contiguous nucleotide sequence is complementary to SEQ ID NO: 11, or a fragment thereof.

24. The oligonucleotide progranulin agonist of embodiment 21, wherein the contiguous nucleotide sequence is complementary to SEQ ID NO: 17, or a fragment thereof.

25. The oligonucleotide progranulin agonist of embodiment 21, wherein the contiguous nucleotide sequence is complementary to SEQ ID NO: 18, or a fragment thereof.

26. The oligonucleotide progranulin agonist of any one of embodiments 1 to 25, wherein the contiguous nucleotide sequence is fully complementary to the promoter of the human progranulin gene.

27. The oligonucleotide progranulin agonist of any one of embodiments 1 to 26, wherein the oligonucleotide is a double stranded oligonucleotide.

28. The oligonucleotide progranulin agonist of embodiment 27, wherein the oligonucleotide is a saRNA.

29. The oligonucleotide progranulin agonist of embodiment 27 or embodiment 28, wherein the sense strand of the contiguous nucleotide sequence is a sequence selected from the group consisting of SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, and SEQ ID NO: 79, or at least 10 contiguous nucleotides thereof.

30. The oligonucleotide progranulin agonist of embodiment 29, wherein the sense strand of the contiguous nucleotide sequence is a sequence selected from the group consisting of SEQ ID NO: 26, SEQ ID NO: 30, SEQ ID NO: 40, SEQ ID NO: 42, SEQ ID NO: 44, SEQ ID NO: 46, SEQ ID NO: 52, SEQ ID NO: 56, SEQ ID NO: 58, SEQ ID NO: 62, SEQ ID NO: 64, SEQ ID NO: 68, SEQ ID NO: 72, and SEQ ID NO: 79, or at least 10 contiguous nucleotides thereof.

31. The oligonucleotide progranulin agonist of embodiment 30, wherein the sense strand of the contiguous nucleotide sequence is a sequence selected from the group consisting of SEQ ID NO: 40, SEQ ID NO: 44, SEQ ID NO: 56, SEQ ID NO: 58, and SEQ ID NO: 79 or at least 10 contiguous nucleotides thereof.

32. The oligonucleotide progranulin agonist of embodiment 31, wherein the sense strand of the contiguous nucleotide sequence is SEQ ID NO: 40, or at least 10 contiguous nucleotides thereof.

33. The oligonucleotide progranulin agonist of embodiment 31, wherein the sense strand of the contiguous nucleotide sequence is SEQ ID NO: 44, or at least 10 contiguous nucleotides thereof.

34. The oligonucleotide progranulin agonist of embodiment 31, wherein the sense strand of the contiguous nucleotide sequence is SEQ ID NO: 56, or at least 10 contiguous nucleotides thereof.

35. The oligonucleotide progranulin agonist of embodiment 31, wherein the sense strand of the contiguous nucleotide sequence is SEQ ID NO: 58, or at least 10 contiguous nucleotides thereof.

36. The oligonucleotide progranulin agonist of embodiment 27 or embodiment 28, wherein the antisense strand of the contiguous nucleotide sequence is a sequence selected from the group consisting of SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73,and SEQ ID NO: 82, or at least 10 contiguous nucleotides thereof.

37. The oligonucleotide progranulin agonist of embodiment 36, wherein the antisense strand of the contiguous nucleotide sequence is a sequence selected from the group consisting of SEQ ID NO: 27, SEQ ID NO: 31, SEQ ID NO: 41, SEQ ID NO: 43, SEQ ID NO: 45, SEQ ID NO: 47, SEQ ID NO: 53, SEQ ID NO: 57, SEQ ID NO: 59, SEQ ID NO: 63, SEQ ID NO: 65, SEQ ID NO: 69, SEQ ID NO: 73, and SEQ ID NO: 82, or at least 10 contiguous nucleotides thereof.

38. The oligonucleotide progranulin agonist of embodiment 37, wherein the antisense strand of the contiguous nucleotide sequence is a sequence selected from the group consisting of SEQ ID NO: 41, SEQ ID NO: 45, SEQ ID NO: 57, SEQ ID NO: 59, and SEQ ID NO: 82, or at least 10 contiguous nucleotides thereof.

39. The oligonucleotide progranulin agonist of embodiment 38, wherein the antisense strand of the contiguous nucleotide sequence is SEQ ID NO: 41, or at least 10 contiguous nucleotides thereof.

40. The oligonucleotide progranulin agonist of embodiment 38, wherein the antisense strand of the contiguous nucleotide sequence is SEQ ID NO: 45, or at least 10 contiguous nucleotides thereof.

41. The oligonucleotide progranulin agonist of embodiment 38, wherein the antisense strand of the contiguous nucleotide sequence is SEQ ID NO: 57, or at least 10 contiguous nucleotides thereof.

42. The oligonucleotide progranulin agonist of embodiment 38, wherein the antisense strand of the contiguous nucleotide sequence is SEQ ID NO: 59, or at least 10 contiguous nucleotides thereof.

43. The oligonucleotide progranulin agonist of any one of embodiments 1 to 26, wherein the oligonucleotide is a single stranded oligonucleotide.

44. The oligonucleotide progranulin agonist of embodiment 43, wherein the oligonucleotide is an antisense oligonucleotide.

45. The oligonucleotide progranulin agonist of embodiment 43 or embodiment 44, wherein the contiguous nucleotide sequence is complementary to the sense strand of the human progranulin gene.

46. The oligonucleotide progranulin agonist of embodiment 45, wherein the contiguous nucleotide sequence is selected from the group consisting of SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, and SEQ ID NO: 82, or at least 10 contiguous nucleotides thereof.

47. The oligonucleotide progranulin agonist of embodiment 46, wherein the contiguous nucleotide sequence is selected from the group consisting of SEQ ID NO: 27, SEQ ID NO: 31, SEQ ID NO: 41, SEQ ID NO: 43, SEQ ID NO: 45, SEQ ID NO: 47, SEQ ID NO: 53, SEQ ID NO: 57, SEQ ID NO: 59, SEQ ID NO: 63, SEQ ID NO: 65, SEQ ID NO: 69, SEQ ID NO: 73, and SEQ ID NO: 82, or at least 10 contiguous nucleotides thereof.

48. The oligonucleotide progranulin agonist of embodiment 47, wherein the contiguous nucleotide sequence is a sequence selected from the group consisting of SEQ ID NO: 41, SEQ ID NO: 45, SEQ ID NO: 57, SEQ ID NO: 59, and SEQ ID NO: 82, or at least 10 contiguous nucleotides thereof.

49. The oligonucleotide progranulin agonist of embodiment 48, wherein the contiguous nucleotide sequence is SEQ ID NO: 41, or at least 10 contiguous nucleotides thereof.

50. The oligonucleotide progranulin agonist of embodiment 48, wherein the contiguous nucleotide sequence is SEQ ID NO: 45, or at least 10 contiguous nucleotides thereof.

51. The oligonucleotide progranulin agonist of embodiment 48, wherein the contiguous nucleotide sequence is SEQ ID NO: 57, or at least 10 contiguous nucleotides thereof.

52. The oligonucleotide progranulin agonist of embodiment 48, wherein the contiguous nucleotide sequence is SEQ ID NO: 59, or at least 10 contiguous nucleotides thereof.

53. The oligonucleotide progranulin agonist of embodiment 43, wherein the contiguous nucleotide sequence is complementary to the antisense strand of the human progranulin gene.

54. The oligonucleotide progranulin agonist of embodiment 53, wherein the contiguous nucleotide sequence is selected from the group consisting of SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 79, or at least 10 contiguous nucleotides thereof.

55. The oligonucleotide progranulin agonist of embodiment 54, wherein the contiguous nucleotide sequence is selected from the group consisting of SEQ ID NO: 26, SEQ ID NO: 30, SEQ ID NO: 40, SEQ ID NO: 42, SEQ ID NO: 44, SEQ ID NO: 46, SEQ ID NO: 52, SEQ ID NO: 56, SEQ ID NO: 58, SEQ ID NO: 62, SEQ ID NO: 64, SEQ ID NO: 68, SEQ ID NO: 72, SEQ ID NO: 79, or at least 10 contiguous nucleotides thereof.

56. The oligonucleotide progranulin agonist of embodiment 55, wherein the contiguous nucleotide sequence is selected from the group consisting of SEQ ID NO: 40, SEQ ID NO: 44, SEQ ID NO: 56, SEQ ID NO: 58, and SEQ ID NO: 79, or at least 10 contiguous nucleotides thereof.

57. The oligonucleotide progranulin agonist of embodiment 56, wherein the contiguous nucleotide sequence is SEQ ID NO: 40, or at least 10 contiguous nucleotides thereof.

58. The oligonucleotide progranulin agonist of embodiment 56, wherein the contiguous nucleotide sequence is SEQ ID NO: 44, or at least 10 contiguous nucleotides thereof.

59. The oligonucleotide progranulin agonist of embodiment 56, wherein the contiguous nucleotide sequence is SEQ ID NO: 56, or at least 10 contiguous nucleotides thereof.

60. The oligonucleotide progranulin agonist of embodiment 56, wherein the contiguous nucleotide sequence is SEQ ID NO: 58, or at least 10 contiguous nucleotides thereof.

61. The oligonucleotide progranulin agonist of any one of embodiments 1 to 60, wherein the oligonucleotide is or comprises an oligonucleotide mixmer or totalmer.

62. The oligonucleotide progranulin agonist of any one of embodiments 1 to 61, wherein the oligonucleotide progranulin agonist is covalently attached to at least one conjugate moiety.

63. The oligonucleotide progranulin agonist of any one of embodiments 1 to 62, wherein the oligonucleotide progranulin agonist is in the form of a pharmaceutically acceptable salt.

64. The oligonucleotide progranulin agonist of embodiment 63, wherein the salt is a sodium salt or a potassium salt.

65. A pharmaceutical composition comprising the oligonucleotide progranulin agonist of any one of embodiments 1 to 64 and a pharmaceutically acceptable diluent, solvent, carrier, salt and/or adjuvant.

66. The pharmaceutical composition of embodiment 65, wherein the pharmaceutical composition comprises an aqueous diluent or solvent, such as phosphate buffered saline.

67. An in vivo or in vitro method for upregulating or restoring progranulin expression in a target cell, said method comprising administering the oligonucleotide progranulin agonist of any one of embodiments 1 to 64, or the pharmaceutical composition of embodiment 65 or embodiment 66, in an effective amount, to said cell.

68. The method of embodiment 67, wherein the cell is either a human cell or a mammalian cell.

69. A method for treating or preventing a disease comprising administering a therapeutically or prophylactically effective amount of the oligonucleotide progranulin agonist of any one of embodiments 1 to 64, or the pharmaceutical composition of embodiment 65 or 66, to a subject suffering from or susceptible to a disease.

70. The method of embodiment 69, wherein the disease is a neurological disease.

71. The method of embodiment 70, wherein the neurological disease is a TDP-43 pathology.

72. The method of embodiment 70, wherein the disease is progranulin haploinsufficiency.

73. The method of embodiment 70, wherein the disease is selected from the group consisting of frontotemporal dementia (FTD), amyotrophic lateral sclerosis (ALS), frontotemporal dementia with neuropathologic frontotemporal lobar degeneration (FTLD), familial frontotemporal dementia with neuropathologic frontotemporal lobar degeneration associated with accumulation of TDP-43 inclusions (FTLD-TDP), and neuronal ceroid lipofuscinosis (NCL).

74. The oligonucleotide of any one of embodiments 1 to 64, or the pharmaceutical composition of embodiment 65 or embodiment 66, for use in medicine.

75. The oligonucleotide progranulin agonist of any one of embodiments 1 to 64, or the pharmaceutical composition of embodiment 65 or embodiment 66, for use in the treatment or prevention of a disease.

76. The oligonucleotide progranulin agonist for use according to embodiment 75, wherein the disease is a neurological disease.

77. The oligonucleotide progranulin agonist for use according to embodiment 76, wherein the neurological disease is a TDP-43 pathology.

78. The oligonucleotide progranulin agonist for use according to embodiment 75, wherein the disease is progranulin haploinsufficiency.

79. The oligonucleotide progranulin agonist for use according to embodiment 75, wherein the disease is selected from the group consisting of frontotemporal dementia (FTD), amyotrophic lateral sclerosis (ALS), frontotemporal dementia with neuropathologic frontotemporal lobar degeneration (FTLD), familial frontotemporal dementia with neuropathologic frontotemporal lobar degeneration associated with accumulation of TDP-43 inclusions (FTLD-TDP), and neuronal ceroid lipofuscinosis (NCL).

80. Use of the oligonucleotide progranulin agonist of any one of embodiments 1 to 64, or the pharmaceutical composition of embodiment 65 or embodiment 66, for the preparation of a medicament for treatment or prevention of a disease.

81. Use of the oligonucleotide progranulin agonist according to embodiment 80, wherein the disease is a neurological disease.

82. Use of the oligonucleotide progranulin agonist according to embodiment 81, wherein the neurological disease is a TDP-43 pathology.

83. Use of the oligonucleotide progranulin agonist according to embodiment 80, wherein the disease is progranulin haploinsufficiency.

84. Use of the oligonucleotide progranulin agonist according to embodiment 80, wherein the disease is selected from the group consisting of frontotemporal dementia (FTD), amyotrophic lateral sclerosis (ALS), frontotemporal dementia with neuropathologic frontotemporal lobar degeneration (FTLD), familial frontotemporal dementia with neuropathologic frontotemporal lobar degeneration associated with accumulation of TDP-43 inclusions (FTLD-TDP), and neuronal ceroid lipofuscinosis (NCL).

### EXAMPLES

### Example 1: Effect of saRNAs on Progranulin mRNA

Materials and Methods:

The day before transfection, H4 neuroglioma cells (n=4) were plated 50000 pr well in 48 well plates in full growth medium (DMEM Sigma: D0819, 15% FBS, 1 mM Sodium Pyruvate, 25 μg/ml Gentamicin). The day after plating, the cells were either untransfected or transfected with saRNA SEQ ID NO: 2, SEQ ID NO: 3, SEQ ID NO: 4, or SEQ ID NO: 5 (n=4) or PBS (Mock) using Lipofectamin RNAiMax (Invitrogen) for final concentration of 10 nM according to Invitrogen's instructions. Three days after transfection, mRNA were isolated using MagNa Pure 96 Instrument (Roche Life Science) and extracted in 50 μL RNAse free Water. After mRNA dilution 10× in RNAse free Water, 1 μL was used as input for one-step ddPCR analysis according to protocol (One-Step RT-ddPCR Advanced Kit for Probes # 1864022, Bio-Rad) using predesigned qPCR assay GRN (Hs.PT.58.2528960.g, IDT) and HPRT1 (HEX, Hs.PT.58v.45621572, IDT). GRN mRNA concentrations were quantified relative to the housekeeping gene HPRT1 using QuantaSoft Software (Bio-Rad).

Results:

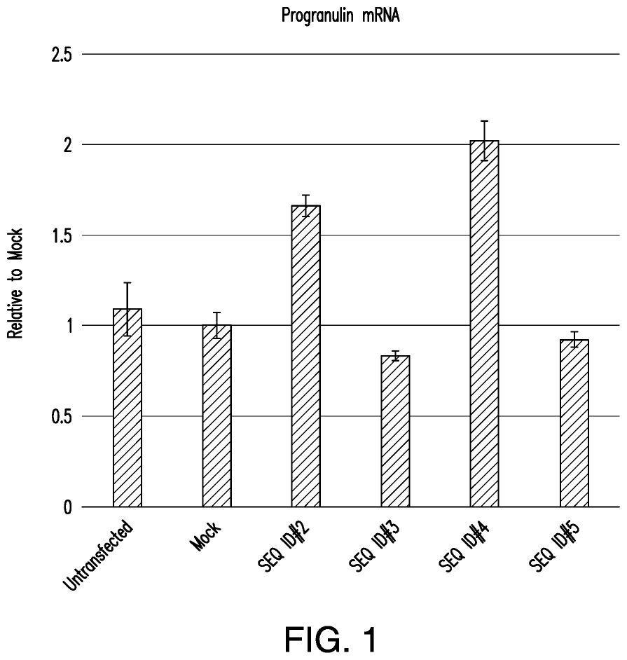

FIG. 1 shows results, wherein SEQ ID NO: 2 and SEQ ID NO: 4 both increased expression of GRN mRNA at 1.7 and 2.0 fold, respectively, relative to Mock transfected 72 h after transfection in H4 cells.

### Example 2: Effect of saRNAs on Progranulin mRNA

Materials and Methods:

The day before transfection H4 neuroglioma cells were plated 15000 per well in 96 well plates in full growth medium (DMEM Sigma: D0819, 15% FBS, 1 mM Sodium Pyruvate, 25 μg/ml Gentamicin). The day after plating, the cells were either untransfected or transfected with saRNA SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, and SEQ ID NO: 25 (n=3) or PBS (Mock) using Lipofectamin RNAiMax (Invitrogen) for final concentration of 10 nM according to Invitrogen's instructions. Three days after transfection, mRNA were isolated using MagNa Pure 96 Instrument (Roche Life Science) and extracted in 50 μL RNAse free Water. After mRNA dilution 10× in RNAse free Water, 1 μL was used as input for one-step ddPCR analysis according to protocol (One-Step RT-ddPCR Advanced Kit for Probes # 1864022, Bio-Rad) using predesigned qPCR assay GRN (Hs.PT.58.2528960.g, IDT) and HPRT1 (HEX, Hs.PT.58v.45621572, IDT). GRN mRNA concentrations were quantified relative to the housekeeping gene HPRT1 using QuantaSoft Software (Bio-Rad).

Results:

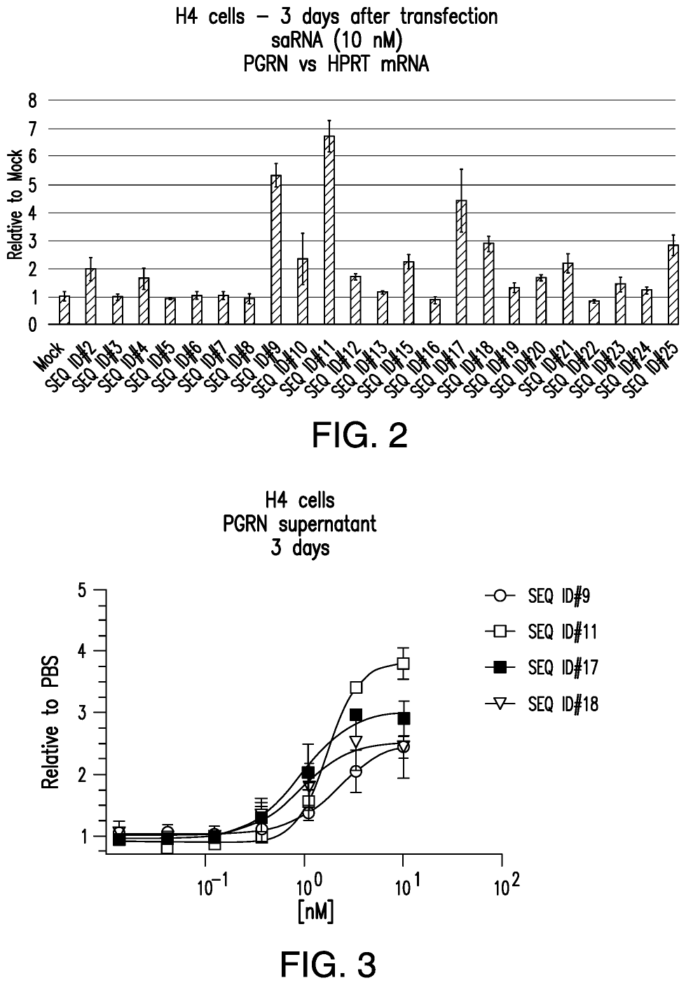

FIG. 2 shows results, wherein SEQ ID NO: 2, SEQ ID NO: 4, SEQ ID NO: 9, SEQ ID NO: 10, SEQ ID NO: 11, SEQ ID NO: 12, SEQ ID NO: 15, SEQ ID NO: 17, SEQ ID NO: 18, SEQ ID NO: 20, SEQ ID NO: 21, SEQ ID NO: 23 and SEQ ID NO: 25 all increased expression of GRN mRNA more than 1.5 fold relative to Mock transfected 72 h after transfection in H4 cells.

### Example 3: Effect of saRNAs on Secreted Progranulin

Materials and Methods:

The day before transfection H4 neuroglioma cells were plated 15000 per well in 96 well plates in full growth medium (DMEM Sigma: D0819, 15% FBS, 1 mM Sodium Pyruvate, 25 μg/ml Gentamicin). The day after plating, the cells were either untransfected or transfection with saRNA SEQ ID NO: 9, SEQ ID NO: 11, SEQ ID NO: 17 and SEQ ID NO: 18 (n=3) or PBS (Mock) using Lipofectamin RNAiMax (Invitrogen) for final indicated concentrations according to Invitrogen's instructions. Three days after transfection Progranulin protein was measured in the supernatant. Progranulin protein expression levels were evaluated in the media after dilution 1:8 by ELISA from Abcam (ab252364) according to manufacturer's protocol. Progranulin levels were normalized to PBS treated cells and values above >1 therefore indicates an upregulation of PGRN protein levels.

Results:

FIG. 3 shows results, wherein SEQ ID NO: 9, SEQ ID NO: 11, SEQ ID NO: 17 and SEQ ID NO: 18 increased expression of progranulin protein in a dose dependent manner 72h after transfection in H4 cells

### Example 4: Upregulation of Mature GRN mRNA

Materials and Methods:

The mRNA sequence upregulated following treatment with saRNAs targeting the progranulin promoter was investigated to determine whether upregulated mRNA comprised a full length GRN mRNA sequence.

The day before transfection H4 neuroglioma cells (n=4) were plated 50000 pr well in 48 well plates in full growth medium (DMEM Sigma: D0819, 15% FBS, 1 mM Sodium Pyruvate, 25 μg/ml Gentamicin). The day after plating, the cells were either untransfected or transfected with saRNA SEQ ID NO: 9, SEQ ID NO: 11, SEQ ID NO: 17 and SEQ ID NO: 18 (n=3) or PBS (Mock) using Lipofectamin RNAiMax (Invitrogen) for final concentration of 1 nM according to Invitrogen's instructions. Three days after transfection, mRNA was isolated using the MagNa Pure 96 Instrument (Roche Life Science) and extracted in 50 μL RNAse free Water.

Samples were DNase treated and total RNA isolated using a MagNA Pure system. Next generation sequencing (NGS) libraries were prepared from 100 ng of the total RNA mixture, using a KAPA Stranded mRNA library system. Samples were sequenced using a NextSeq 550 system, to obtain approximately 25 million PE reads (2×151 bp). Data analysis was performed using CLC Genomics Workbench 20 (Qiagen). Reads were trimmed to remove the last base at the 3′ end, and reads below 100 nucleotides were removed prior to transcriptome analysis. Samples were subsampled to 15 million reads prior to mapping. RNA-Seq analysis was performed using CLC Genomic workbench, with sequences mapped to Genome Reference Consortium Human Reference 38, hg38. The BAM file format was used for input into Sashimi plots.

Results:

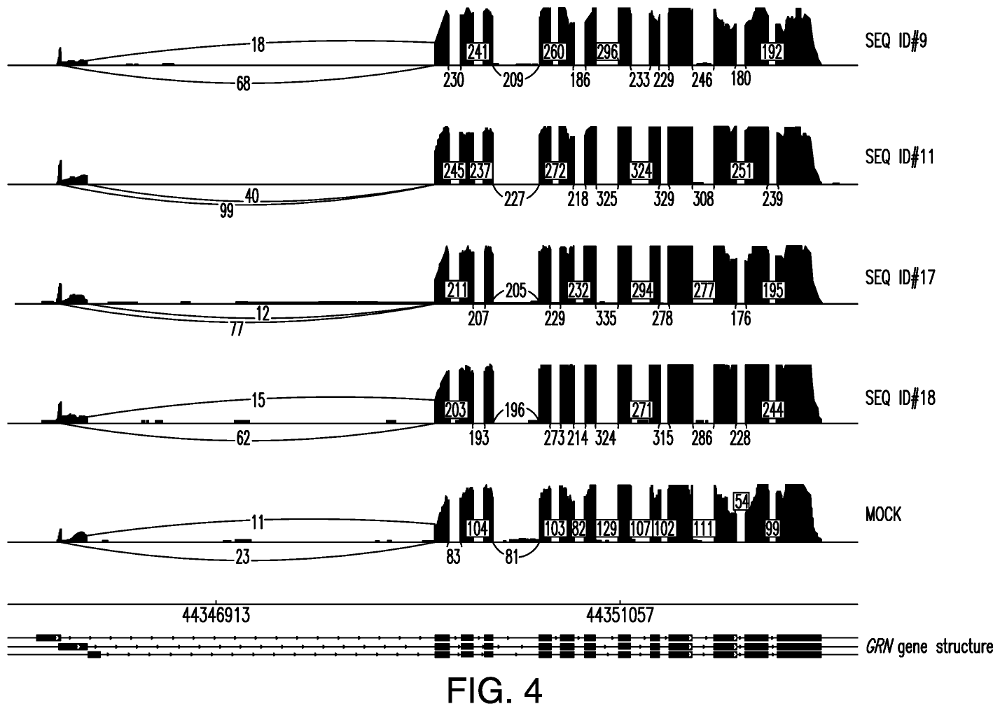

Treatment of cells with saRNAs targeting the promoter region of the GRN gene resulted in upregulation of mature GRN mRNA (as shown in Table 2 & FIG. 4). The Sashimi plot (FIG. 4) demonstrates an increase in the number of reads corresponding to each of the 13 exons that comprise a GRN mRNA. These data indicate that treatment with the saRNAs of the invention resulted in upregulation of the entire mature GRN mRNA, which would then be available for translation into progranulin. As detailed in Table 2, samples SEQ ID NO: 9, SEQ ID NO: 11, SEQ ID NO: 17 and SEQ ID NO: 18, (saRNAs 8, 10, 16, and 17, respectively) yielded a 2.4, 2.9, 2.5 and 2.6 fold increase of GRN exon 1-13 spanning reads, respectively, as compared to the MOCK control.

TABLE 2
Exon spanning reads of GRN mRNA following oligonucleotide transfection.

| | | | | | | | | | | | | | | Sum of | Fold |
|---|---|---|---|---|---|---|---|---|---|---|---|---|---|---|---|
| Sample ID | | Exon junction (Number of exon spanning reads in | | | | | | | | | | | | GRN exon | increase |
| (target | | GRN per 15 million reads) | | | | | | | | | | | | spanning | compared |
| sequence) | saRNA | 1-2 | 2-3 | 3-4 | 4-5 | 5-6 | 6-7 | 7-8 | 8-9 | 9-10 | 10-11 | 11-12 | 12-13 | reads (1-13) | to MOCK |
| SEQ ID NO: 9 | 8 | 68 | 230 | 241 | 209 | 260 | 186 | 296 | 233 | 229 | 246 | 180 | 192 | 2570 | 2.4 |
| SEQ ID NO: 11 | 10 | 99 | 245 | 237 | 227 | 272 | 218 | 325 | 323 | 329 | 308 | 251 | 239 | 3073 | 2.9 |
| SEQ ID NO: 17 | 16 | 77 | 213 | 207 | 205 | 229 | 232 | 335 | 294 | 278 | 277 | 176 | 195 | 2718 | 2.5 |
| SEQ ID NO: 18 | 17 | 62 | 203 | 193 | 196 | 273 | 214 | 324 | 273 | 315 | 286 | 228 | 244 | 2811 | 2.6 |
| MOCK | n/a | 23 | 83 | 104 | 81 | 103 | 82 | 129 | 107 | 102 | 111 | 54 | 99 | 1078 | 1.0 |

### Example 5: Specificity of saRNAs on GRN mRNA

Materials and Methods:

The day before transfection, SK-N-AS neuroblastoma cells were plated to a density of 25000 per well in 96 well plates in full growth medium (DMEM (Sigma: D6546), 10% FBS, 2 mM glutamine, 0.1 mM (1×) NEAA, 25 μg/ml Gentamicin). The day after plating, the cells were either transfected with saRNA SEQ ID NO: 9, SEQ ID NO: 79, SEQ ID NO: 80, SEQ ID NO: 81 (n=3) or PBS using Lipofectamin RNAiMax (Invitrogen) at a final concentration of 20, 10, 3.3, 1.11, 0.37, 0.12, or 0.004 nM according to Invitrogen's instructions. 48 h after transfection, mRNA were isolated using the RNeasy® 96 Kit (Qiagen) and extracted in 200 μL RNAse free Water. 4 μL was used as input for one-step RT-qPCR analysis according to the protocol gScript™ XLT One-Step RT-qPCR ToughMix®, Low ROX™, Quanta Bioscience, #95134-500 using qPCR assays specific to GRN (Hs.PT.58.2528960.g, IDT) and HPRT1 (HEX, Hs.PT.58v.45621572, IDT). GBA mRNA concentrations were quantified relative to the housekeeping gene HPRT1 using R Software.

Results:

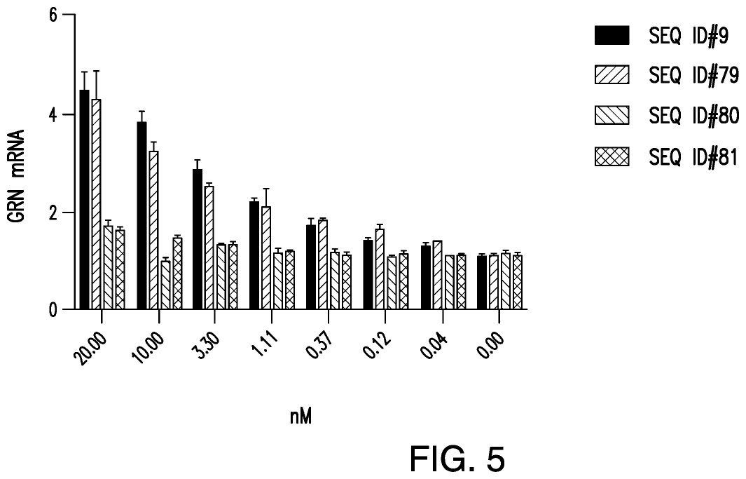

Targeting SEQ ID NO: 9 (i.e. saRNA formed from SEQ ID NO: 40 and SEQ ID NO: 41) and SEQ ID NO: 79 (i.e. saRNA formed from SEQ ID NO: 79 and SEQ ID NO: 82) increased GRN mRNA dose dependently 48 h after transfection in SK-N-AS cells. Mutations in the seed region of the antisense strand SEQ ID NO: 80 (i.e. saRNA formed from SEQ ID NO: 80 and SEQ ID NO: 83) and SEQ ID NO: 81 (i.e. saRNA formed from SEQ ID NO: 81 and SEQ ID NO: 84) abolished the dose dependent upregulation of GRN mRNA, as shown in FIG. 5.

That which is claimed is:

1. An oligonucleotide progranulin agonist, wherein the oligonucleotide is 8-40 nucleotides in length and comprises a contiguous sequence of 8-40 nucleotides in length, which is complementary to the promoter of the human progranulin gene.

2. The oligonucleotide progranulin agonist of claim 1, wherein the contiguous nucleotide sequence is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length, and wherein the oligonucleotide is the same length as the contiguous nucleotide sequence.

3. The oligonucleotide progranulin agonist of claim 1, wherein the promoter of the human progranulin gene comprises a promoter selected from the group consisting of SEQ ID NO: 76, SEQ ID NO: 75, SEQ ID NO: 1, SEQ ID NO: 77, SEQ ID NO: 78, and SEQ ID NO: 74

4. The oligonucleotide progranulin agonist of claim 1, wherein the contiguous nucleotide sequence is complementary to a sequence selected from the group consisting of nucleotides 131 to 151 of SEQ ID NO: 1, nucleotides 231 to 251 of SEQ ID NO: 1, nucleotides 568 to 588 of SEQ ID NO: 1, nucleotides 783 to 803 of SEQ ID NO: 1, nucleotides 9 to 30 of SEQ ID NO: 1, nucleotides 29 to 49 of SEQ ID NO: 1, nucleotides 50 to 70 of SEQ ID NO: 1, nucleotides 107 to 127 of SEQ ID NO: 1, nucleotides 163 to 183 of SEQ ID NO: 1, nucleotides 199 to 219 of SEQ ID NO: 1, nucleotides 272 to 292 of SEQ ID NO: 1, nucleotides 296 to 316 of SEQ ID NO: 1, nucleotides 403 to 423 of SEQ ID NO: 1, nucleotides 481 to 501 of SEQ ID NO: 1, nucleotides 520 to 540 of SEQ ID NO: 1, nucleotides 597 to 617 of SEQ ID NO: 1, nucleotides 685 to 705 of SEQ ID NO: 1, nucleotides 809 to 829 of SEQ ID NO: 1, nucleotides 844 to 864 of SEQ ID NO: 1, nucleotides 876 to 896 of SEQ ID NO: 1, nucleotides 905 to nucleotides 925 of SEQ ID NO: 1, nucleotides 965 to 985 of SEQ ID NO: 1, nucleotides 1004 to 1024 of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, and any fragments thereof.

5. The oligonucleotide progranulin agonist of claim 1, wherein the contiguous nucleotide sequence is fully complementary to the promoter of the human progranulin gene.

6. The oligonucleotide progranulin agonist of claim 1, wherein the oligonucleotide is a double stranded oligonucleotide, such as wherein the oligonucleotide is a saRNA.

7. The oligonucleotide progranulin agonist of claim 6, wherein the sense strand of the contiguous nucleotide sequence is selected from the group consisting of SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 79, and any sequence of at least 10 contiguous nucleotides thereof.

8. The oligonucleotide progranulin agonist of claim 6, wherein the antisense strand of the contiguous nucleotide sequence is selected from the group consisting of SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 82, and any sequence of at least 10 contiguous nucleotides thereof.

9. The oligonucleotide progranulin agonist of claim 1, wherein the oligonucleotide is a single stranded oligonucleotide, such as wherein the oligonucleotide is an antisense oligonucleotide.

10. The oligonucleotide progranulin agonist of claim 9, wherein the contiguous nucleotide sequence is complementary to the sense strand of the human progranulin gene, and wherein the contiguous nucleotide sequence is selected from the group consisting of SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 82, and any sequence of at least 10 contiguous nucleotides thereof.

11. The oligonucleotide progranulin agonist of claim 9, wherein the contiguous nucleotide sequence is complementary to the antisense strand of the human progranulin gene, and wherein the contiguous nucleotide sequence is selected from the group consisting of SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 79, and any sequence of at least 10 contiguous nucleotides thereof.

12. The oligonucleotide progranulin agonist of claim 1, wherein the oligonucleotide progranulin agonist is an oligonucleotide mixmer or totalmer, further comprising:
a. the oligonucleotide progranulin agonist being covalently attached to at least one conjugate moiety; and,
b. the oligonucleotide progranulin agonist being in the form of a pharmaceutically acceptable salt.

13. A pharmaceutical composition comprising the oligonucleotide progranulin agonist of claim 1 and a pharmaceutically acceptable diluent, solvent, carrier, salt and/or adjuvant, wherein the pharmaceutical composition comprises an aqueous diluent or solvent.

14. An in vivo or in vitro method for upregulating or restoring progranulin expression in a target cell, said method comprising administering the oligonucleotide progranulin agonist of claim 1 in an effective amount to said cell, wherein the cell comprises a human cell or a mammalian cell.

15. The oligonucleotide progranulin agonist of claim 1 for use in the treatment or prevention of a disease, wherein the disease is selected from a group consisting of a TDP-43 pathology, a progranulin haploinsufficiency, frontotemporal dementia (FTD), amyotrophic lateral sclerosis (ALS), frontotemporal dementia with neuropathologic frontotemporal lobar degeneration (FTLD), familial frontotemporal dementia with neuropathologic frontotemporal lobar degeneration associated with accumulation of TDP-43 inclusions (FTLD-TDP), and neuronal ceroid lipofuscinosis (NCL).


SEQUENCE LISTING (84 sequences)

SEQ ID NO: 1 - DNA, 1207 bp, Artificial Sequence
  Synthetic polynucleotide
ccagttaaaatcttcccagactcagctcaaggagatgctcctaaggtggaatgaaatctcttcttccccacctggagacaatctacttcctctccctacacctggcaactggcgcacaaccttgtatcttaaattagattcagcctgagactgtctcccaccaatccctgctccctgtcctgctgagcaccttgaggaaagggctttggggctgtttatctttgtcctggaaaccatccttcaactcactctggggcctgcctagcatgtcaaccgagtttggagaatagggcagaatagggcaggacaggacaggacaagacagggcaggataggataggagcgagccagctcagtagctcacatttgtaatcccagcgccttggggggctgcggtaggagaatcgctttgggagcaggagttgcaggccgcagtgagctatgatcagcttgggcgactgagcgagaccctgtctctaaaacaaacacacaagtccgggcgcggtggctcatgcctgtaatcttagcactttgggaggccgaggtgggcggatcacgaggtcaagaaatcgagaccatcctggccaacatggtgaaaccccgtctctactaaaaatacaaaaattagctgggcgtggtggtgcgcgcctgtagtcccagctactcgggaggctgaggcaggagaatcgcttgaacccgggaggcagaggttgcagtgagccgagatcgtgccactgcactccagcctggcgacagagtgagactccgtctcagaacaaacaaacaaaaggatagaaaggcgagcacaaatattcccaattcataacactccctcgcactgtcaatgccccagacacgcgctatcatctctagcaaactcccccaggcgcctgcaggatgggttaaggaaggcgacgagcaccagctgccctgctgaggctgtcccgacgtcacatgattctccaatcacatgatccctagaaatggggtgtggggcgagaggaagcagggaggagagtgatttgagtagaaaagaaacacagcattccaggctggccccacctctatattgataagtagccaatgggagcgggtagccctgatccctggccaatggaaactgaggtaggcgggtcatcgcgctggggtctgtagtctgagcgctacccggttgctgctgcccaaggaccgcggagtcggacgcagg

SEQ ID NO: 2 - DNA, 21 bp, Artificial Sequence
  Synthetic polynucleotide
aaattagattcagcctgagac

SEQ ID NO: 3 - DNA, 21 bp, Artificial Sequence
  Synthetic polynucleotide
aaaccatccttcaactcactc

SEQ ID NO: 4 - DNA, 21 bp, Artificial Sequence
  Synthetic polynucleotide
aatcgagaccatcctggccaa

SEQ ID NO: 5 - DNA, 21 bp, Artificial Sequence
  Synthetic polynucleotide
aacaaaaggatagaaaggcga

SEQ ID NO: 6 - DNA, 22 bp, Artificial Sequence
  Synthetic polynucleotide
aatcttcccagactcagctcaa

SEQ ID NO: 7 - DNA, 21 bp, Artificial Sequence
  Synthetic polynucleotide
aaggagatgctcctaaggtgg

SEQ ID NO: 8 - DNA, 21 bp, Artificial Sequence
  Synthetic polynucleotide
aatgaaatctcttcttcccca

SEQ ID NO: 9 - DNA, 21 bp, Artificial Sequence
  Synthetic polynucleotide
aactggcgcacaaccttgtat

SEQ ID NO: 10 - DNA, 21 bp, Artificial Sequence
  Synthetic polynucleotide
aatccctgctccctgtcctgc

SEQ ID NO: 11 - DNA, 21 bp, Artificial Sequence
  Synthetic polynucleotide
aagggctttggggctgtttat

SEQ ID NO: 12 - DNA, 21 bp, Artificial Sequence
  Synthetic polynucleotide
aaccgagtttggagaataggg

SEQ ID NO: 13 - DNA, 21 bp, Artificial Sequence
  Synthetic polynucleotide
aatagggcaggacaggacagg

SEQ ID NO: 14 - DNA, 3 bp, Artificial Sequence
  Synthetic polynucleotide
  misc_feature (1)..(3): n is a, c, g, or t
nnn

SEQ ID NO: 15 - DNA, 21 bp, Artificial Sequence
  Synthetic polynucleotide
aatcgctttgggagcaggagt

SEQ ID NO: 16 - DNA, 21 bp, Artificial Sequence
  Synthetic polynucleotide
aacaaacacacaagtccgggc

SEQ ID NO: 17 - DNA, 21 bp, Artificial Sequence
  Synthetic polynucleotide
aatcttagcactttgggaggc

SEQ ID NO: 18 - DNA, 21 bp, Artificial Sequence
  Synthetic polynucleotide
aaccccgtctctactaaaaat

SEQ ID NO: 19 - DNA, 21 bp, Artificial Sequence
  Synthetic polynucleotide
aatcgcttgaacccgggaggc

SEQ ID NO: 20 - DNA, 21 bp, Artificial Sequence
  Synthetic polynucleotide
aatattcccaattcataacac

SEQ ID NO: 21 - DNA, 21 bp, Artificial Sequence
  Synthetic polynucleotide
aatgccccagacacgcgctat

SEQ ID NO: 22 - DNA, 21 bp, Artificial Sequence
  Synthetic polynucleotide
aactcccccaggcgcctgcag

SEQ ID NO: 23 - DNA, 21 bp, Artificial Sequence
  Synthetic polynucleotide
aaggaaggcgacgagcaccag

SEQ ID NO: 24 - DNA, 21 bp, Artificial Sequence
  Synthetic polynucleotide
aatcacatgatccctagaaat

SEQ ID NO: 25 - DNA, 21 bp, Artificial Sequence
  Synthetic polynucleotide
aagcagggaggagagtgattt

SEQ ID NO: 26 - DNA, 21 bp, Artificial Sequence
  Synthetic polynucleotide
auuagauucagccugagactt

SEQ ID NO: 27 - DNA, 21 bp, Artificial Sequence
  Synthetic polynucleotide
gucucaggcugaaucuaautt

SEQ ID NO: 28 - DNA, 21 bp, Artificial Sequence
  Synthetic polynucleotide
accauccuucaacucacuctt

SEQ ID NO: 29 - DNA, 21 bp, Artificial Sequence
  Synthetic polynucleotide
gagugaguugaaggauggutt

SEQ ID NO: 30 - DNA, 21 bp, Artificial Sequence
  Synthetic polynucleotide
ucgagaccauccuggccaatt

SEQ ID NO: 31 - DNA, 21 bp, Artificial Sequence
  Synthetic polynucleotide
uuggccaggauggucucgatt

SEQ ID NO: 32 - DNA, 21 bp, Artificial Sequence
  Synthetic polynucleotide
caaaaggauagaaaggcgatt

SEQ ID NO: 33 - DNA, 21 bp, Artificial Sequence
  Synthetic polynucleotide
ucgccuuucuauccuuuugtt

SEQ ID NO: 34 - DNA, 22 bp, Artificial Sequence
  Synthetic polynucleotide
ucuucccagacucagcucaatt

SEQ ID NO: 35 - DNA, 22 bp, Artificial Sequence
  Synthetic polynucleotide
uugagcugagucugggaagatt

SEQ ID NO: 36 - DNA, 21 bp, Artificial Sequence
  Synthetic polynucleotide
ggagaugcuccuaagguggtt

SEQ ID NO: 37 - DNA, 21 bp, Artificial Sequence
  Synthetic polynucleotide
ccaccuuaggagcaucucctt

SEQ ID NO: 38 - DNA, 21 bp, Artificial Sequence
  Synthetic polynucleotide
ugaaaucucuucuuccccatt

SEQ ID NO: 39 - DNA, 21 bp, Artificial Sequence
  Synthetic polynucleotide
uggggaagaagagauuucatt

SEQ ID NO: 40 - DNA, 21 bp, Artificial Sequence
  Synthetic polynucleotide
cuggcgcacaaccuuguautt

SEQ ID NO: 41 - DNA, 21 bp, Artificial Sequence
  Synthetic polynucleotide
auacaagguugugcgccagtt

SEQ ID NO: 42 - DNA, 21 bp, Artificial Sequence
  Synthetic polynucleotide
ucccugcucccuguccugctt

SEQ ID NO: 43 - DNA, 21 bp, Artificial Sequence
  Synthetic polynucleotide
gcaggacagggagcagggatt

SEQ ID NO: 44 - DNA, 21 bp, Artificial Sequence
  Synthetic polynucleotide
gggcuuuggggcuguuuautt

SEQ ID NO: 45 - DNA, 21 bp, Artificial Sequence
  Synthetic polynucleotide
auaaacagccccaaagccctt

SEQ ID NO: 46 - DNA, 21 bp, Artificial Sequence
  Synthetic polynucleotide
ccgaguuuggagaauagggtt

SEQ ID NO: 47 - DNA, 21 bp, Artificial Sequence
  Synthetic polynucleotide
cccuauucuccaaacucggtt

SEQ ID NO: 48 - DNA, 21 bp, Artificial Sequence
  Synthetic polynucleotide
uagggcaggacaggacaggtt

SEQ ID NO: 49 - DNA, 21 bp, Artificial Sequence
  Synthetic polynucleotide
ccuguccuguccugcccuatt

SEQ ID NO: 50 - DNA, 3 bp, Artificial Sequence
  Synthetic polynucleotide
  misc_feature (1)..(3): n is a, c, g, or t
nnn

SEQ ID NO: 51 - DNA, 3 bp, Artificial Sequence
  Synthetic polynucleotide
  misc_feature (1)..(3): n is a, c, g, or t
nnn

SEQ ID NO: 52 - DNA, 21 bp, Artificial Sequence
  Synthetic polynucleotide
ucgcuuugggagcaggagutt

SEQ ID NO: 53 - DNA, 21 bp, Artificial Sequence
  Synthetic polynucleotide
acuccugcucccaaagcgatt

SEQ ID NO: 54 - DNA, 21 bp, Artificial Sequence
  Synthetic polynucleotide
caaacacacaaguccgggctt

SEQ ID NO: 55 - DNA, 21 bp, Artificial Sequence
  Synthetic polynucleotide
gcccggacuuguguguuugtt

SEQ ID NO: 56 - DNA, 21 bp, Artificial Sequence
  Synthetic polynucleotide
ucuuagcacuuugggaggctt

SEQ ID NO: 57 - DNA, 21 bp, Artificial Sequence
  Synthetic polynucleotide
gccucccaaagugcuaagatt

SEQ ID NO: 58 - DNA, 21 bp, Artificial Sequence
  Synthetic polynucleotide
ccccgucucuacuaaaaautt

SEQ ID NO: 59 - DNA, 21 bp, Artificial Sequence
  Synthetic polynucleotide
auuuuuaguagagacggggtt

SEQ ID NO: 60 - DNA, 21 bp, Artificial Sequence
  Synthetic polynucleotide
ucgcuugaacccgggaggctt

SEQ ID NO: 61 - DNA, 21 bp, Artificial Sequence
  Synthetic polynucleotide
gccucccggguucaagcgatt

SEQ ID NO: 62 - DNA, 21 bp, Artificial Sequence
  Synthetic polynucleotide
uauucccaauucauaacactt

SEQ ID NO: 63 - DNA, 21 bp, Artificial Sequence
  Synthetic polynucleotide
guguuaugaauugggaauatt

SEQ ID NO: 64 - DNA, 21 bp, Artificial Sequence
  Synthetic polynucleotide
ugccccagacacgcgcuautt

SEQ ID NO: 65 - DNA, 21 bp, Artificial Sequence
  Synthetic polynucleotide
auagcgcgugucuggggcatt

SEQ ID NO: 66 - DNA, 21 bp, Artificial Sequence
  Synthetic polynucleotide
cucccccaggcgccugcagtt

SEQ ID NO: 67 - DNA, 21 bp, Artificial Sequence
  Synthetic polynucleotide
cugcaggcgccugggggagtt

SEQ ID NO: 68 - DNA, 21 bp, Artificial Sequence
  Synthetic polynucleotide
ggaaggcgacgagcaccagtt

SEQ ID NO: 69 - DNA, 21 bp, Artificial Sequence
  Synthetic polynucleotide
cuggugcucgucgccuucctt

SEQ ID NO: 70 - DNA, 21 bp, Artificial Sequence
  Synthetic polynucleotide
ucacaugaucccuagaaautt

SEQ ID NO: 71 - DNA, 21 bp, Artificial Sequence
  Synthetic polynucleotide
auuucuagggaucaugugatt

SEQ ID NO: 72 - DNA, 21 bp, Artificial Sequence
  Synthetic polynucleotide
gcagggaggagagugauuutt

SEQ ID NO: 73 - DNA, 21 bp, Artificial Sequence
  Synthetic polynucleotide
aaaucacucuccucccugctt

SEQ ID NO: 74 - DNA, 20391 bp, Artificial Sequence
  Synthetic polynucleotide
gcccgccgcgggccgaccagccgcaagcccggagtgtccccggctccgcccactcgcgtctccgcccggacctgcgacggtcccgcccccttgacggggccgcgcgggcgagttccatgcctgtccagcgctgagagctgccggccaacttcgcggtctccacgtcgcgcttaacgtggcagcagcacgcacttccagcatctttttttagacagcatctttttcagcctcgctctgtcgccgaggctggagggcagtggcgcgatctaggctcactgcagccttgacctcccgggttcaagcgatcctcctgcctcagcctccggagtagctgggaccacaggcgcgcgccgccacgcccggctggtttgttacttttatttttgtctccctattttgcccaggctagtctggaactcctgggctcaagcgatccgcccacctcggtcttcgaaagtgctgggattccaggcgtgagccgccgcgccggccttaacagcgcattttcaccagccccgccccggccgcgcgcggcactggaaaccccagcgccttctggacggcgagggtgacgcactggccctcttcctcggtgtcctcaggtgcgtggcggcggcagtgagaatgagttcttcgagatcgccgggagaccacacaataacgacgaagttacttgccgggctacccgccgagacttgaaaacttcgcggcgacttctctcgccggccttagcatcgcccccatttttcaggttacaggacaggcctgcaactgcaaaacgctttgtaaaccacagggccccgcgcgggtgtgcgccactgggttagctgagctgcagcccgggccgggcgggctgggacgtgagcaagccgggggagacgggagagccatggggagccagatcccaccttcgcgtgaccttgggcaaatcacaacctcgcctaaggcccggaatgactctgcgcggagaggaagagcactgagtgcgggagggcatcgacgttttggtgttggttgctctcgtatcgtgatttcttctgagtctcaaaactctttgcagtaggttattgttgaaatagattaccacaggccgggcgcggtggctcacgcctgtaatccagcactttaggaggccgaggcgggcggatcacttgaagtcaggagttcgagaccagcctggccaacatggtgaaaccccgtctctactaaaaatacaaaagtagccgggcatgatggctggcgcctgtaatcccagctactcgggaggctgaggcaggagaatcgcttgaacccggccggcggaggttgcagtgagccgagatcgcaccactgcattccagcctgggcaacagagtaacactccgtctcaaaaaaaaaggccgggcgcagtggcttacgcctgtaatcccagcctttttggaggccgaggcgggcggatcaccagaggtcaggagttcgagagcagcctgaccaacatggtgaaacctcgtctctactaaaaatacaaaaaattagccgggcgtggtggcgcatgccagtaatcccagccactcgagaggctgaggcaggagaatcgcttaaacccggaaggcggaggttgcggtgagccgagactgcgccattgcactccagcctgggcaacaagagcgaaactccgtctgaaaaagaaatagattaccccgttttacagaaagtgaaactgaggctcgacgtgaagagccggagattcgaaccggccgggtgcttttcccagggctccacactgcctccccgggagacaggaaggcttaagtccagcgctgccctctccgatcccgcttgtcagggagacactttatttcccggggcagccccgtaccccaggccccaccacccacttcgcgtttagaacttctgtgctatcttgcttatttgtcctaacagagaaaccagcacgcgcgggcggtggcggaggggggcgggcgggcgggtggaggcgggggggggcgggcggggagatgggtactcctagcctgcgcctttaagaagggtaggcctcgactttgacgtctctgccttccccgcccttcaggcccccactggtcgcgtccggcgctggaggagcccagtcagccggcgcctgccgggttagcacgtggactccgaggggccaactatcagctttccctgacaaaatgcctttgagctcccccacagctctgaaactccagcttgggagcagggagagtgcaatctgtgacctgtaaaggggcgctgggcaaaagggcccgagagaaggcgtcctttccattcccttcccatctcaagctgagttcagaaatgacacgaaataatttaatcaactatcagtcaaggtcgggctcacgcctgtaatcccagcagtttgggaggccgaggtgggcggatcacctgaggacaggagttcgagaccagcctgaccaacatgacgaagccccgtctctactaaaaatacaaaaattagccagcggtggtggtgcgcacctgtagtctcagctacttgggaggctgaggccagagaatcgcttgagcccgcgaggtcgaggctgcagtgagccgagatcacaccactgcactccaacctggatgacagagggagactctgtctcaaaaaataataaataaataaataaataaataaactatcggtcaaggtgaaagcctactctccgccaaggttctttacaacttagacgttttggtttacggaacaggggaggaaactgagggctcttgtgtgtagtgactttactcgtgtggaggagaacccagactttccctgcacacactctcccgttttatggatgagaaaaggggggccccaaaggtcaaatcgccccaaggtcactcaaatgaaagagtcagcattagaccccccaacgtgcctgagccagatgggctcccaccctgtccagttctcaacccaggtgctgtgttccagcagcaccctgacctggctttccatctcacctgcctcttacaagcaagtgtcttaaggcctctaagccttagtttccccatttacaaaatgggctgatggtgatgataacgcaccttcctctagggattatgtaaggatttaatgcattaatggctgtgacgtgcttagaaccctggcaagtggcaagcattctatgacaactactactattgctgttactgtattgttgttaattgctctctagaacctgtggcatggaaagttccccagattctggccccacctctgcctccagtatgcagtgatggataataataacttcttatacccagaacgaaggtcaccagtgcaaagtagcacagccaggaatcaaacctggggacctctgagcctgtttccccaacagtacattgagggccttgcactccatttctggttttctttttgtgatgctcttaaataggatccttagagatttgtggatacacctgtttcctcatctgaaaataaggacgatatcacctgctctgcctaacgtacagattcttaatatcatgtctctgaaagctacatgaagagtcaataaacagttctgtgagacctgccaggggcccagagccgcgtggatcacagtccttaccttctccagaatgtaaatgctttgaagcttagagaaccatgaggcctcatgggtgtgaccaaagcagtggccgctggagaggttatccatgggtgatgggacacctcgggacctcgtgaagtctctcccaagaatgaccgtggcagttcagggtggaggaggagctttgacatgctccagaagctatggagccctgactcaacacgcccaccctcgaggagcaagcctggaaggctgcagaggtggccactggcctggctgactcaggcaaggtttacattggttatgcacccagtggtccttccagaggcaacatgtggccccatgtggccatctgcagcttcctcgcacagatggaaaagcagaaactctccctttttaactgtaaaaggggagaagaaagtaccagctgcaggggtggttgtggagactgaaggaccagtcctcacctgatacccaaggggccagtttattacaaggctgaatttgtccaaggcaagccctgggacaggggcagtgtcccctccagcttgaacccccagggcctaggaagacatctctggggctgatggaggactcaagggcttcacccatggatgggaccccaccagcacctctgagtccagggccagcactgtctgcatctagagtagtctcccaaagcagggcccagggctgcagaaccactgaggccccctactgcccagaggaaaagtccaaatcccaggcccatagcaaatgtagtctgatcttttttttttttttttttttttttttgagacggagtctcgctctgtcgcccaggctggactgcagactgcagtggcgcaatctcggctcactgcaagctccgcttcccgggttcacgccattctcctgcctcagcctcccgagtagctgggactacaggcgcccgccaccgcgcccggctaattttttgtatttttagtagagacggggtttcaccttgttagccaggatggtctcgatctcctgacctcatgatccacctgcctcggcctcccaaagtgctggaattacaggcgtgagccaccgcgcccggcctgtagtctgatctttaatggattctgagggtggaaaggagtaggggagtgatggaggactttattggaacaagtggagaaaagggaataaggtctgtatgctaattactcttttttttttttgagacgaagtcttgctctgtaccacaatgcccggctaatttttttttgtttatttatttatttatttatttatttatttatttatttatttatttttggagagaccgggttttccatgttggccaggctggtctcaaactcctgacctcaggtgatccacccaccttggcctcccaaagtgctgggatttcaggcatgagccaccatgcctggcctcccaggttcaagcaattctcctgtctcagcctcccgagtagctgggattacaggtatgcaccaccatgcctggccaatttttgtattctcagtagagacagggttttgcaatgtaggccgggctggtcttgaactactgacttcaggtgatccacccacctcggcctcccaaagtgctggaattataggcgtgagccacagcacctggccaatattactttatcagtgttaaaatttctggagtgataacaatactgtgattttgtaggagaatgccctgttcttaggaaacacatactgtagtgtttagggatgaataaagtgttgtgatgtctgtaacttagtcctaaatggctcagcaaaaaaagaagccaggcatggtagctcatgcctgtaatggaggctgaggcagcaggaggatcgcttgagcccaggagtttgagaccagcctgggcaacatagggagaccctgtctcttataaaaaacaaaaaaaaaattaactgggtgtggtgttgtgcacctgtagtctcagctacttgggaggctgaggtgggaggattgcttgagcccatgaggtggaggctacagtgagcagtgattgtgccactgcactccagcctgggtgacagagcaagatcttatctcaaaaaaaaaaaaaaaaaaaaagccagtgtagcaaaatgttagccccatctctactaaaaatacaaaaattagccagccgtggttgcgggtgcctataatcccagctactcaggaggctgaggcgggagaatcgcttgaacccaggaggtggaggtcacagtgagctgagatcacaccactgcactccagcctgggtgacagagcgagactccgtctcaaagaaaaaaaaaaaagaagatatatatatatatatatatgcatacgtgtgtgtatgtgtgtgagtgtatatatatgtatatttgtgtgtgtgtgtgtgtgtatatatatatatatatatatatatatatatatatatatatatatataaaacagctgggatcagcccaatttggggaagtcctccctgtctccccctgcctctgtggcctgagaaggtgggctggaagtgtctaggcacccaaggctttattggggggccctgggtgtggtctgaggaggtgggcccctagagggagagggtcctggctaagactttccagcatgtcccccagcctgggtcccagagctctgctgggacagcgtggattaatatggaaggtagttttagccatagctatgaaaattctaaacgccaaacccttgatccagccaggccacttctaagcgtttctctagtcatctgctcatacaagtgtgaaatgatggtaccagcttggccttcttcattgcagccttctttgtaacagcagaacatcccgtgaaagggaactgttaagtacgtgaccctatctccatgtgacaaatccacacagccttgaaagggcggcattccctgacctatccaggtggggtggggggtgagggctgcagatctctatatcattatgtaaaacaatctcaaagacagagttaaattaaacagcaggtgctgagagcgtgctggacactcgttccaaacagcagcgttactgccccctagaggacattttttgaaatttatgcggccactgtcatttctggttgtcacaagactggggcttctggcatttgatagatggcaggtcagggaagctaaatgccctgtaaggtgtaggacagtctgacacaatagggaatttccaacaacaagaacacatctctcaatttccttctgtctttctgggcaattacatacatgaggaaatgtttgcaattctcagacctggaaaatggttctgttttacccatatacccacacctttttttttttttttagacagggtctctcactctgtcgctggttggagtgcagtagcacgatcacagctcactacagcctcgacctccccggctcaagcgatcctcccacctcagcctcccgagtagcacactcctctgcctgactaattcgtttgtatttgttgtagagacagggtttcgctatgctgcccaggttgtaaccatacttcttttgaatacttatattctgctatgcattgagttttccaggaataaaaccactctgcagttgtatggaaaattgggttttgtcttcaagaactttaccgaaggtcatttgccatttcattttttgtttggtttgttttgtttttttgagatggagttttgctcttgtcgcccaggctggagtgcagtggtacgatcttggctcactgtaacctctgcctcctgggttcaagcaattctcctgcctcagcctcccaagtacacgcccagctaattttgtatttttagtagagacagggtttcctcatgttggtcaggctggtctcaaactcccgacctcaggtaatctgtctgcctcggcctcccaaagtgctgggattacaggtgtgagccaccgtgcctggcctttttttttttttttttttttaagacgggacctcactgttgctcaggctggagtgcagtggtgtgatcatagctcactgcagcattgaatccctgagctcaagtgatcctagtgcttcaccttctcaagtagctgggactacaggagcgtgccacctcgcctggctaatacatatatatatatatttttttgagtcagggtctctcactctgttgcccaggctggagtgccgtggcacaatcacagctcgctgcagcctcgacgtcctggattaaagcgatcctcccacctcagctctctcccccctccccccaccagtagctgggaccacagtcacgcaccaccacgcccagctaatttttgtttattttttatagagaaggggtgtcagtatgttgcccacgatggtctcaaactcctgggctcatgtgatcctcctgcatccacctcccaaagtgtcaggattacaccacagggcccagccttagtcatttcactgtaagatgggaataataattgtactacctcaaaggattgtgttacagattaaagagcataatacagaacctggcacaatataaatctcaataaacagtaacaggacctaccttttaaaaacactgaaatgaagtatgccagctatcatcctagcagaagagattgaattaaagtccccttatatgtatttgagtacagatttctctgctaattcagagaattaggggaccttgtctccatcctgggtctcagtttcttcatctttaaaatggaaaactgggtgtggtggctcacgcctataatcccagcactttgggagcccaacgtgggcagatcacctgaggccaggagttcgagaccagcctgatcaacatggtgaaaccccatctctactaaaaatgcaaaaattagctgggcgtggtggtgcacacctgtaatcccagctacttgggaggctgaggcacaagaatcgcttgaacctgagaggcagagggtgcagtgagctgaaatcacaccactgcactccaacctgggcaacagagcgattctctgtctcaaaaataaataaataaccaaattaaaataaaaaataaaataaaattgaaaggatagtactcctggccccacttccttcctagggctgccaaggtctaagctgagagatgaggggtgtggcaacagtgcatgacagggaaagctggcctggggcgggccgctccttctcccttcctgtcctccgtgcagctgacttctccctgcccttctctgggttctggagatgactctgacccaacctctctgagctctctgagtggggcagcttcctcacctaggccctggcctattgcttcagagacaggtccccaccgggttaacaagaggaacccctttgatgtcctaaaaagaattcctcctgtttttaatgtgccagtttacaagatctcttcacatccatcatcccgtttgaacctcacatgactgtgtggagggcaagcgggggttattgtgtacacattttacagatgaggaaaccgagtcttggaggagttgagtaatccattcaagtcagttggagcagacatctcctgactcacagggggcccacctggagaaaggccaggggcactcaggtgggctggcagggctgggagggctgggagggaggatcagggccgtgagctgagctggcatcggcatccagtggagctggtgaggtgggcaaaactctgggctctgattgatttgcactcagacaccaacaccactgcatcctagctaatgactggataagactttagcatctcatatgcctgtgaatggagatcattctctggagtgttacaggaactctgcatttgaaacacaggcctgattcttgccatctctactcctgctcccagctctcacagccagagtcaacagtgcccgcctgtccttccactgccaccacccaaactgtctacctgctgcccttaacaacagataggatccaactcctagagtccccaagcaattcagggaccactcagaggtgaaacaaccccaaccattctcaaccttagcaaaaggaacagggtggtgaagggtactccccatgcccatagggacacaggacactggccagaccaccaagtggcgcctggccccccatgggccttcctgacccggtagccacccctcccaggggaaggacacagtgttctcagatgtgggcccaggggctgtgtcatggaggagctgggaagggtttagaggacctgggtttctgtctttggtttttttttttctttccactctagtaaaatcaaattttatatcattgattttgtaaagtttaattggaaaatgaatttctttgtgttgaataagtgcaatttgctggtcaaaattcattcaaacataggagtgagagaggaggaagtgactgtttattgaggaactgtgatgtgccaggcactccacaagatgttttgctcattcaggcctcctgagcaccctgggagacaggcattccaatgagcccatttcacagatgaaggcatttagggtcaaaacaataagccaaagtcacccagctggcaggaggccacgctggaatccaagctcagcagacagtgaagaggcccgtttgctctgctcccagcctgtctgctaggcttgcagaaaagcactgactcttgtcctctggcacctgccacctgcctgtctggggtgggaaaggcgagaggacaagggtgctgctttttgttataatttgattaaaatgcatttgtggccgggcgcggtggctcatgcctgtgatcccagcactttgggaggctgaggcaggcggatcgcttgagctcaagagtttgagacctgcctgggcaacatagtgaaaccctgtctctaccaaaaatacaaaaaagtagccaggcgtgttggtgtgtacctgtggtcccagctacttaggaagctgaggcagggggatcgcttgaacctggaaggtggaggttgcagtgagctgagatcgcgccactgcactccagcctgggtgagcgagaccgtgtctcaaaaaaaaaaaaaaaaagtatttgcatatgacactttgttaaaacagtgccatggctcacgcttgtaatcccagcattttaggaggatcacttgagcccaggagttcgagaccagcttgggcaatacaaccagaccccatctctaccaaaaaataaaataaaattagtcggacacggtggtgtgcacctgtagtcccagctactctggggacttaagtgggaggatcaattgagctcaggagttcaaggctgcagtgagccatgatcataccactgtactccagcttaggtgacagagtaagaccctgtgtctaaaaagcaaacaaacaaaaaacaaacagttattgagtgaaaaacaaaacagagaagtggcttatccactgtcactgggcaagttcacggcttatgctgggatgggaacatgggtctcctgactgtggtcagtacttctgttcccaaataccctgccacagcctctagcctgggcactgggctgttcctgcttctcctccctcattacccccccatagcccagtaaccccccacccctagctctggtttccctttctgcctaaagggagacgctctgtcctgatatggagcattccccccaggacccccagccctaccactcatgagttggaatagttccgaaaggacatagcctgtctgctcctgctatctctgcctgccgtgtggggagctggtccagaatgatggagcccctggggagcaaggggtcgtgtccagcttgggagggcttctggagacctggcccgagtcccagatccttcttgaactagaaaaatgcctgctgtttttagtgcattctcagttgacaaagcattttcttacctctaatcccattggatctttataatgaagaaggaattccctgtcaatctgcattgtattttatttaattaatttatttattggttttgagacagagtctcgctctgttgcccaggctggagtgcggtggcatgatctcgcctcactgcaacctccgcctcctggttcaagcaattctagtgcctcagcctctcaagtagctgggattataggggtgtgccaccacacctggctaatttttttaaaaaatttacttatttattttgagacggaatcttgctctgtcgcccagactagagtgcagtggcgtgatctcagctcactgtaacctccatctcctgggttcaactgattctctcacctcagcttcccgagtagctgggattacaggcgcccgccaccatgcccggctaatttttgtatttttagtagagatgaggtttcaccatgttagccaggctgctatcgaactcctgacctcaaatgatctggctgcctcagcccccctgcattgcattttaaatctgagagacagaggctgagagtggcctgtgtctagcaccaggacaggctacacagtttggaggtccagtgcaaatgaaaatgtgggtctccttgttcaaaagtattaggactctggccaggtgcgatggctcacgcctgtaatctcagcactttgggaggctgaggcggatggatcacctgagatcaggagtttgagaccagcctggccaatgtggtgaaagcccgtctctactaaaaatacaaaaattagctgggcatggtggcagatgcctgtaatctcagctactcaggaggctgaggcaggagaagcacttgaacccaggaggcggaggttgcagtgaaccgagatcacgccattgcactccagcctgggtgacatataaggtccagccctacggggcttagcaggtgttctccccgtgtgcggagatgagagatcataagaaataaagacacaagacaaagagataaagagaaaacagctggaccctggggaccactaccaccaagacgcggagaccggtagtggccccgaatggctgggggcactgacatctattgcatacaagacaaggggggcagggtaaggagggtgagtcgtccaagcgattgataaggtcaagcaagtcaagtgatcatgggatagggggcccttcccttttaggtagccaaagcagagagggaaggcagcatatgtcagcgttttcttctatgcatgtatcaaaaagatcaaagactttaaggctttcactatttcttctaccactatctactacgaacttcaaagaggaatccaggagtatgggaggaacatgaaagtggacaaggagcgtgaccactgaagcacagcaccacagaaaggggtttaggccttgggatgactgcgggcaggcctggataatatccagcctcccacaagaagctggcggagcagagtgtttcctgactcctcgaaggaaaggagactccctttcttggtctgctaagtagcgggtgccttcccaggcactggcgttaccgcttgaccaaggagccctcaagcggcccttatgagggcgtgacagagggctcacctcttgccttcttggtcacttctcataatgtcccttcagcacctgaccctataccctccggttattccttggttatattagtaatacaacaaagagtcatattaaaggctaataattaataatgtctatactaatgattgataatgcccatgaccatctctatatctaatttgtattataactattctcattctaactattttctttattatactgaaacagtctgtgccttcaatctcttgcctcggcacctgggtaatcctccgcccacagtgacaagagtgaaactctgtcttaaaaaaaaaaattaagaatttcaagataggacagcagagctttaaattttttttttttcctagaggcagagtcttgctctcttgcccaggctggggtgcattgttgcaatcacatatcactgcagccttgagctcccgggctcaagtggtcatcctgctttggcctcccaaagtcctgggattacaggcgtgagccactgcacccggcctgtagagctttaaacaaagcacaggctgcttctgagcacagggccctcgtcacatggctatgaagccagccctgcctggacccagaaccctggtgtgggcactttatagcttataagccctccagtcaagaggcctcagcctgagcctccctgagcctcagtttcctctcctgtaatctgtgaaagcactgagcactaatctcccttccgggctagctttctgcaatagcttaaagctcccatcccaagatctctccaagaatcctcttaccaagtccctgctcccagcctggctccctggtccccatccctctcccctctcacaccctttcaaagcccagaaaggacaggtgaccgagtgactgtctctgggctcatacagtggggtggtctcctgtgacaggctagggcttcagggtatggcttagcctatccctcacacttccttggggcctcttggggcagacagcccaggcggggcccagcaggcacactagtcaggtgcctgatgatgagggcaggcgcctggaactgggtcaacaatcgctcacacggaggtgactcaagcagcaaggggaaaaagaggtgggggtggggaggtggctgtttctgtgccctcacttgagcctcagcttttagggttgccagatttatttttatttttatttttgagagtctcactctgtctcccaggctggagtgcagtggcgcaatcttggctcactgcaacctctgcctcccaggttcaagcaattctcgtgcctcagcctcccaagtagctgggattacagatgtgcaccaccacccctggctaatttttgtatttttagtagagatggggtttcaccatgttggccagtctggttttgaactcctaacctcaggtgatccacccacctcagcctcccaaagtgctgggattataggcgtgagccaccattcctggcagcctgaaaattttttaatttttttttaatttttttttttttttagtgatggggtctccatatgttgcccaggctggtctcaaactcctaggctcaagtgatcctttcaccttggccttggcctcctaaagtgctgggattacaggcgtgacccactgtgcctgcctgctggtatgtaacttgttgttttttttttttttttagacaaagtctcacttgccctggctcaagtgcagtggtgccatcttggctcactgcagtctccgcctcccaggttcaagtgattctcctccctcagcctcctgagtagctgggattacaggcatgtgccaccatgcccagctaatttttgtatttttagtagagacagggtttcaccatgctggccaggctggtcttgaactcctaaccttgagtgatctacccacctctgcctcccaaagtgctggattacaggccaataggcctggccccctgtaacttttttttttttttttaatttttgagacagagtcttgctctgtcgcccaggctggagtgcagtggtgccatctcggctcactgcaagctccgcctccctggttcacgccattctcctgcctcagcctcccgagtagctgggactacaggcgcctaccaccacgcctggctaattttttggatttttagtagagatggagtttcaccgtgttagccaggatggtctccatttcctgaccttgtgatccacccgcctcagcctcccaaagtgctgggattacaggcgtgagccaccacgcccggcccctgtaacttcttaagcgaaggaaaataaaaaaaaaaaaaaagcagaagccacaaaggaaaagattgaccaatatatttcagatgacataacaacaacacaaaagacaaatgacagattgggaggaagcatctgaaacatacaaaatacccagaatatataaacagctcctacagttcaacaagaaagaaaatactgcaaaaacaatcagcaaataatactgtgatggtagaacaatgtatctacttggatggtgctcctggattagattaacatttaaatcagtgaactttggttaaagcaaattgctctccattgtagggagaccccctgaaactattgctacggaataaaagatgaaatgctcctgattattgtaaatacgaaattgcatgcaggattgtgtaaagacaatgccaggttggactgccagtataagccaacagctcgtgatgtgcttccccctgaagagagcctatgaacagacgtgcagtcagggaggtttcacatcaccaatattcctatcccagaaaagcagatgttcatagccctgggaatggaatgtgacccttgtggagggcctataaatggacgcatgagcggcacctgttcatatggataagatagggctataaatgccctcatcttgccatggctcttctaggcctctttaaggttaaggcatactcccttctgataatttctggtctaactggttatctagcttcacgtcctgtttctatggattgtttgtaaccagcttttgctgcaactcttactgctgattaatatcttgctaatcataggttatggaaagactgtgtttctgttttaaggctctgttagaaattactgatgcacacactatattgtaaattcttatctctgtatactgtacttctgcatacagatgttatgttaaagaattacttcatccccatgtgaccatctcacctcataatcaaacgaccctaaatccctcactaacctaccctcgccctcactaaacttaataataaatgctggtatatccagtgcattggcagcatcgcaggaccagaaggcggtgacccccttggacccagctttcactatcttgtgtgtgtctattatttcccgacctgccgatccacctggggacaaagagagagccccattgccttgtgggctgctggccagatcccgcgatactccataatgtggatggacctgaataaaacagaaagacgggcctcgccgaactagagggaattctcctgcaggctaccctcagacttaaccatcagctctcctgggtcgccagcccaccagctcacactaaaggcttaggatggttatgataagaaagacagatgataacagaagttaaccatggatgaggctaaagagaaattgaaaccctcatacatcgctggtgggaatgtaaaatagtgcagctgctttggaaaacaggctggtagttacacaaaaggctaaacagaattattatgtaatccactaattctactcctaggtatatacccaagagaaatgaaaacatgtccacacaaaaacttgtatgggccgggcgtggtggctcactcctgtaatcccagccctttgggaggctgaagtgggaggatccctggagttcaggagtttgagactagctgaccaacatggcaagaccctgtctctacaaaaaccacacaaaaattagctggacatggtgataagcacctgtagtcccagctacttgggaggcggaggtgggaggattacttgagcccaggaggcagaggttgcagtgagcagagatgacaccactgcactccaacctgggtaacagagcaataccctgtctcaaaaaaaaaaaaaaaaatgctggggggatgatgaagataggcacagtggctcaagcctgtaatctgtaatctcagcattttgggaggctgaggcagcaggatcacttgaggccaggagcttgagaccagactaggcaacatattaagacaaaaaaatacaaaatatagctgggagtggtggcatgtgtctgtattcccagctactcaggaggctgaggtgggagggtctgaggctgcagtacaaggtggcaccactgtaatccagcttgggcggcagaggaaggctctgtctcaaaaacaaagaaacaaacaaaaatgggccacgcgtggtggctcacgcctgtaatcccagcagtctgggagattgaggtgggtggatcacttgaggtcaggagttcgagaccagcctggtcaacatggcggaaccctgtctctactaaaatacaaaaattagctgggcatcatggcacatgcttgtaatcccagctatccgggaggctgagacaggagaattgcttgaacccaggaggcagaggttgcagtgagccaagatcgtgccactgcactccagcttgagtgttggagtgagactctgtctcaaacaaatgaaaagaaacaaacaaaaaagaaatgagatactgatacctgctatgtgataagctttgaaaacattatgctaagtgaaagaagccagttgcagatcacatattataaaatttcatttatatggaatatccaaatctatagaggtagaaagtagatcagtggttgcctagggttagggagggtgactgctaaaggacataaggctgccttctgtggtgataaaaatggtcttaaactgattgtaatgatgctgcataactctcttaagacactaaaaaccattgagttgtacactttaatttttttgtgttggcgggggtggatggagtctcgctctgttgtccaggctggaatgcagtggcacgatctcggtttactgcaacctctgcatcccgggttcaagcgattctcctgcctcggcctcctgagtagggaattacagacctcgttatcgtggcaccttacccttctgatgttaaaaaaaaaaaaaaaaagagcgagagagagagagagaaacatttgtgaagtaggttgttgagtctcagcactattgaccttttgggcaggatacttctttgttgtgggggattgttctgtgtgtcgtgtgatgtttagtgggattgctggcccttacctaccagatgccagtgtccctccaccctgagttgtgacaacccagattgtctccagacactcctaaatgtccctggccggcaaaattgccgctgctcaagaatcacggctttgacgattagactttgtgatatttgtttcagtctgtttaggttttttttcttctacctgtatttttttctggttctgggtggttgtaattagtaggttattgatcgattcacctaacatttcatgaaagtttcatgtgtgtgtgtgtttcaatagaagcataaactatactccctagtctcaagatacacaggaaggaaaataagcacaaatgtgtcaccagggcacagactagtactaggtcctcagcaggccaggtgtcttatccgctgtctgggtctgctctagctccaggcttagaacccctgccacacgactccacagctcggttggcaccctttccctcctccgacttctgctgcctcgagcttggttagccatccccctgcccctgcctcatcctcagctccagttccttgctcaggctgcagcagtctccatcccctgtgcagacactgccgttcctccacggcccagtatcaggctttccctgggcctctcctctctcctggcccatctcccatcatccatctctgcctggcccaggccctttggcaccaagcaggctgactcttgtcactggctaatctgttctgtggtacattttctctcctcaccctcccatatcaattcctcgaaggcagggccgatctggagactaggaagccacttctctttcgacagcccccaccacagcccagcccgtgccaggcacccagcagctcctgaagcccactggcattgaacatggcattcaatccctgccaagcctgcccttcccatctggtttcccagggctcttcccaacacctcctcctccacctgccagttaaaatcttcccagactcagctcaaggagatgctcctaaggtggaatgaaatctcttcttccccacctggagacaatctacttcctctccctacacctggcaactggcgcacaaccttgtatcttaaattagattcagcctgagactgtctcccaccaatccctgctccctgtcctgctgagcaccttgaggaaagggctttggggctgtttatctttgtcctggaaaccatccttcaactcactctggggcctgcctagcatgtcaaccgagtttggagaatagggcagaatagggcaggacaggacaggacaagacagggcaggataggataggagcgagccagctcagtagctcacatttgtaatcccagcgccttggggggctgcggtaggagaatcgctttgggagcaggagttgcaggccgcagtgagctatgatcagcttgggcgactgagcgagaccctgtctctaaaacaaacacacaagtccgggcgcggtggctcatgcctgtaatcttagcactttgggaggccgaggtgggcggatcacgaggtcaagaaatcgagaccatcctggccaacatggtgaaaccccgtctctactaaaaatacaaaaattagctgggcgtggtggtgcgcgcctgtagtcccagctactcgggaggctgaggcaggagaatcgcttgaacccgggaggcagaggttgcagtgagccgagatcgtgccactgcactccagcctggcgacagagtgagactccgtctcagaacaaacaaacaaaaggatagaaaggcgagcacaaatattcccaattcataacactccctcgcactgtcaatgccccagacacgcgctatcatctctagcaaactcccccaggcgcctgcaggatgggttaaggaaggcgacgagcaccagctgccctgctgaggctgtcccgacgtcacatgattctccaatcacatgatccctagaaatggggtgtggggcgagaggaagcagggaggagagtgatttgagtagaaaagaaacacagcattccaggctggccccacctctatattgataagtagccaatgggagcgggtagccctgatccctggccaatggaaactgaggtaggcgg

SEQ ID NO: 75 - DNA, 2601 bp, Artificial Sequence
  Synthetic polynucleotide
tggatggagtctcgctctgttgtccaggctggaatgcagtggcacgatctcggtttactgcaacctctgcatcccgggttcaagcgattctcctgcctcggcctcctgagtagggaattacagacctcgttatcgtggcaccttacccttctgatgttaaaaaaaaaaaaaaaaagagcgagagagagagagagaaacatttgtgaagtaggttgttgagtctcagcactattgaccttttgggcaggatacttctttgttgtgggggattgttctgtgtgtcgtgtgatgtttagtgggattgctggcccttacctaccagatgccagtgtccctccaccctgagttgtgacaacccagattgtctccagacactcctaaatgtccctggccggcaaaattgccgctgctcaagaatcacggctttgacgattagactttgtgatatttgtttcagtctgtttaggttttttttcttctacctgtatttttttctggttctgggtggttgtaattagtaggttattgatcgattcacctaacatttcatgaaagtttcatgtgtgtgtgtgtttcaatagaagcataaactatactccctagtctcaagatacacaggaaggaaaataagcacaaatgtgtcaccagggcacagactagtactaggtcctcagcaggccaggtgtcttatccgctgtctgggtctgctctagctccaggcttagaacccctgccacacgactccacagctcggttggcaccctttccctcctccgacttctgctgcctcgagcttggttagccatccccctgcccctgcctcatcctcagctccagttccttgctcaggctgcagcagtctccatcccctgtgcagacactgccgttcctccacggcccagtatcaggctttccctgggcctctcctctctcctggcccatctcccatcatccatctctgcctggcccaggccctttggcaccaagcaggctgactcttgtcactggctaatctgttctgtggtacattttctctcctcaccctcccatatcaattcctcgaaggcagggccgatctggagactaggaagccacttctctttcgacagcccccaccacagcccagcccgtgccaggcacccagcagctcctgaagcccactggcattgaacatggcattcaatccctgccaagcctgcccttcccatctggtttcccagggctcttcccaacacctcctcctccacctgccagttaaaatcttcccagactcagctcaaggagatgctcctaaggtggaatgaaatctcttcttccccacctggagacaatctacttcctctccctacacctggcaactggcgcacaaccttgtatcttaaattagattcagcctgagactgtctcccaccaatccctgctccctgtcctgctgagcaccttgaggaaagggctttggggctgtttatctttgtcctggaaaccatccttcaactcactctggggcctgcctagcatgtcaaccgagtttggagaatagggcagaatagggcaggacaggacaggacaagacagggcaggataggataggagcgagccagctcagtagctcacatttgtaatcccagcgccttggggggctgcggtaggagaatcgctttgggagcaggagttgcaggccgcagtgagctatgatcagcttgggcgactgagcgagaccctgtctctaaaacaaacacacaagtccgggcgcggtggctcatgcctgtaatcttagcactttgggaggccgaggtgggcggatcacgaggtcaagaaatcgagaccatcctggccaacatggtgaaaccccgtctctactaaaaatacaaaaattagctgggcgtggtggtgcgcgcctgtagtcccagctactcgggaggctgaggcaggagaatcgcttgaacccgggaggcagaggttgcagtgagccgagatcgtgccactgcactccagcctggcgacagagtgagactccgtctcagaacaaacaaacaaaaggatagaaaggcgagcacaaatattcccaattcataacactccctcgcactgtcaatgccccagacacgcgctatcatctctagcaaactcccccaggcgcctgcaggatgggttaaggaaggcgacgagcaccagctgccctgctgaggctgtcccgacgtcacatgattctccaatcacatgatccctagaaatggggtgtggggcgagaggaagcagggaggagagtgatttgagtagaaaagaaacacagcattccaggctggccccacctctatattgataagtagccaatgggagcgggtagccctgatccctggccaatggaaactgaggtaggcgggtcatcgcgctggggtctgtagtctgagcgctacccggttgctgctgcccaaggaccgcggagtcggacgcaggtaggagagcggccgcgcagacctctcgcctgctcctgcccaggggcccgccagggccatgtgagcttgaggttcccctggagtctcagccggagacaacagaagaaccgcttactgaaactccttgggggttctgatacactag

SEQ ID NO: 76 - DNA, 20609 bp, Artificial Sequence
  Synthetic polynucleotide
gcccgccgcgggccgaccagccgcaagcccggagtgtccccggctccgcccactcgcgtctccgcccggacctgcgacggtcccgcccccttgacggggccgcgcgggcgagttccatgcctgtccagcgctgagagctgccggccaacttcgcggtctccacgtcgcgcttaacgtggcagcagcacgcacttccagcatctttttttagacagcatctttttcagcctcgctctgtcgccgaggctggagggcagtggcgcgatctaggctcactgcagccttgacctcccgggttcaagcgatcctcctgcctcagcctccggagtagctgggaccacaggcgcgcgccgccacgcccggctggtttgttacttttatttttgtctccctattttgcccaggctagtctggaactcctgggctcaagcgatccgcccacctcggtcttcgaaagtgctgggattccaggcgtgagccgccgcgccggccttaacagcgcattttcaccagccccgccccggccgcgcgcggcactggaaaccccagcgccttctggacggcgagggtgacgcactggccctcttcctcggtgtcctcaggtgcgtggcggcggcagtgagaatgagttcttcgagatcgccgggagaccacacaataacgacgaagttacttgccgggctacccgccgagacttgaaaacttcgcggcgacttctctcgccggccttagcatcgcccccatttttcaggttacaggacaggcctgcaactgcaaaacgctttgtaaaccacagggccccgcgcgggtgtgcgccactgggttagctgagctgcagcccgggccgggcgggctgggacgtgagcaagccgggggagacgggagagccatggggagccagatcccaccttcgcgtgaccttgggcaaatcacaacctcgcctaaggcccggaatgactctgcgcggagaggaagagcactgagtgcgggagggcatcgacgttttggtgttggttgctctcgtatcgtgatttcttctgagtctcaaaactctttgcagtaggttattgttgaaatagattaccacaggccgggcgcggtggctcacgcctgtaatccagcactttaggaggccgaggcgggcggatcacttgaagtcaggagttcgagaccagcctggccaacatggtgaaaccccgtctctactaaaaatacaaaagtagccgggcatgatggctggcgcctgtaatcccagctactcgggaggctgaggcaggagaatcgcttgaacccggccggcggaggttgcagtgagccgagatcgcaccactgcattccagcctgggcaacagagtaacactccgtctcaaaaaaaaaggccgggcgcagtggcttacgcctgtaatcccagcctttttggaggccgaggcgggcggatcaccagaggtcaggagttcgagagcagcctgaccaacatggtgaaacctcgtctctactaaaaatacaaaaaattagccgggcgtggtggcgcatgccagtaatcccagccactcgagaggctgaggcaggagaatcgcttaaacccggaaggcggaggttgcggtgagccgagactgcgccattgcactccagcctgggcaacaagagcgaaactccgtctgaaaaagaaatagattaccccgttttacagaaagtgaaactgaggctcgacgtgaagagccggagattcgaaccggccgggtgcttttcccagggctccacactgcctccccgggagacaggaaggcttaagtccagcgctgccctctccgatcccgcttgtcagggagacactttatttcccggggcagccccgtaccccaggccccaccacccacttcgcgtttagaacttctgtgctatcttgcttatttgtcctaacagagaaaccagcacgcgcgggcggtggcggaggggggcgggcgggcgggtggaggcgggggggggcgggcggggagatgggtactcctagcctgcgcctttaagaagggtaggcctcgactttgacgtctctgccttccccgcccttcaggcccccactggtcgcgtccggcgctggaggagcccagtcagccggcgcctgccgggttagcacgtggactccgaggggccaactatcagctttccctgacaaaatgcctttgagctcccccacagctctgaaactccagcttgggagcagggagagtgcaatctgtgacctgtaaaggggcgctgggcaaaagggcccgagagaaggcgtcctttccattcccttcccatctcaagctgagttcagaaatgacacgaaataatttaatcaactatcagtcaaggtcgggctcacgcctgtaatcccagcagtttgggaggccgaggtgggcggatcacctgaggacaggagttcgagaccagcctgaccaacatgacgaagccccgtctctactaaaaatacaaaaattagccagcggtggtggtgcgcacctgtagtctcagctacttgggaggctgaggccagagaatcgcttgagcccgcgaggtcgaggctgcagtgagccgagatcacaccactgcactccaacctggatgacagagggagactctgtctcaaaaaataataaataaataaataaataaataaactatcggtcaaggtgaaagcctactctccgccaaggttctttacaacttagacgttttggtttacggaacaggggaggaaactgagggctcttgtgtgtagtgactttactcgtgtggaggagaacccagactttccctgcacacactctcccgttttatggatgagaaaaggggggccccaaaggtcaaatcgccccaaggtcactcaaatgaaagagtcagcattagaccccccaacgtgcctgagccagatgggctcccaccctgtccagttctcaacccaggtgctgtgttccagcagcaccctgacctggctttccatctcacctgcctcttacaagcaagtgtcttaaggcctctaagccttagtttccccatttacaaaatgggctgatggtgatgataacgcaccttcctctagggattatgtaaggatttaatgcattaatggctgtgacgtgcttagaaccctggcaagtggcaagcattctatgacaactactactattgctgttactgtattgttgttaattgctctctagaacctgtggcatggaaagttccccagattctggccccacctctgcctccagtatgcagtgatggataataataacttcttatacccagaacgaaggtcaccagtgcaaagtagcacagccaggaatcaaacctggggacctctgagcctgtttccccaacagtacattgagggccttgcactccatttctggttttctttttgtgatgctcttaaataggatccttagagatttgtggatacacctgtttcctcatctgaaaataaggacgatatcacctgctctgcctaacgtacagattcttaatatcatgtctctgaaagctacatgaagagtcaataaacagttctgtgagacctgccaggggcccagagccgcgtggatcacagtccttaccttctccagaatgtaaatgctttgaagcttagagaaccatgaggcctcatgggtgtgaccaaagcagtggccgctggagaggttatccatgggtgatgggacacctcgggacctcgtgaagtctctcccaagaatgaccgtggcagttcagggtggaggaggagctttgacatgctccagaagctatggagccctgactcaacacgcccaccctcgaggagcaagcctggaaggctgcagaggtggccactggcctggctgactcaggcaaggtttacattggttatgcacccagtggtccttccagaggcaacatgtggccccatgtggccatctgcagcttcctcgcacagatggaaaagcagaaactctccctttttaactgtaaaaggggagaagaaagtaccagctgcaggggtggttgtggagactgaaggaccagtcctcacctgatacccaaggggccagtttattacaaggctgaatttgtccaaggcaagccctgggacaggggcagtgtcccctccagcttgaacccccagggcctaggaagacatctctggggctgatggaggactcaagggcttcacccatggatgggaccccaccagcacctctgagtccagggccagcactgtctgcatctagagtagtctcccaaagcagggcccagggctgcagaaccactgaggccccctactgcccagaggaaaagtccaaatcccaggcccatagcaaatgtagtctgatcttttttttttttttttttttttttttgagacggagtctcgctctgtcgcccaggctggactgcagactgcagtggcgcaatctcggctcactgcaagctccgcttcccgggttcacgccattctcctgcctcagcctcccgagtagctgggactacaggcgcccgccaccgcgcccggctaattttttgtatttttagtagagacggggtttcaccttgttagccaggatggtctcgatctcctgacctcatgatccacctgcctcggcctcccaaagtgctggaattacaggcgtgagccaccgcgcccggcctgtagtctgatctttaatggattctgagggtggaaaggagtaggggagtgatggaggactttattggaacaagtggagaaaagggaataaggtctgtatgctaattactcttttttttttttgagacgaagtcttgctctgtaccacaatgcccggctaatttttttttgtttatttatttatttatttatttatttatttatttatttatttatttttggagagaccgggttttccatgttggccaggctggtctcaaactcctgacctcaggtgatccacccaccttggcctcccaaagtgctgggatttcaggcatgagccaccatgcctggcctcccaggttcaagcaattctcctgtctcagcctcccgagtagctgggattacaggtatgcaccaccatgcctggccaatttttgtattctcagtagagacagggttttgcaatgtaggccgggctggtcttgaactactgacttcaggtgatccacccacctcggcctcccaaagtgctggaattataggcgtgagccacagcacctggccaatattactttatcagtgttaaaatttctggagtgataacaatactgtgattttgtaggagaatgccctgttcttaggaaacacatactgtagtgtttagggatgaataaagtgttgtgatgtctgtaacttagtcctaaatggctcagcaaaaaaagaagccaggcatggtagctcatgcctgtaatggaggctgaggcagcaggaggatcgcttgagcccaggagtttgagaccagcctgggcaacatagggagaccctgtctcttataaaaaacaaaaaaaaaattaactgggtgtggtgttgtgcacctgtagtctcagctacttgggaggctgaggtgggaggattgcttgagcccatgaggtggaggctacagtgagcagtgattgtgccactgcactccagcctgggtgacagagcaagatcttatctcaaaaaaaaaaaaaaaaaaaaagccagtgtagcaaaatgttagccccatctctactaaaaatacaaaaattagccagccgtggttgcgggtgcctataatcccagctactcaggaggctgaggcgggagaatcgcttgaacccaggaggtggaggtcacagtgagctgagatcacaccactgcactccagcctgggtgacagagcgagactccgtctcaaagaaaaaaaaaaaagaagatatatatatatatatatatgcatacgtgtgtgtatgtgtgtgagtgtatatatatgtatatttgtgtgtgtgtgtgtgtgtatatatatatatatatatatatatatatatatatatatatatatataaaacagctgggatcagcccaatttggggaagtcctccctgtctccccctgcctctgtggcctgagaaggtgggctggaagtgtctaggcacccaaggctttattggggggccctgggtgtggtctgaggaggtgggcccctagagggagagggtcctggctaagactttccagcatgtcccccagcctgggtcccagagctctgctgggacagcgtggattaatatggaaggtagttttagccatagctatgaaaattctaaacgccaaacccttgatccagccaggccacttctaagcgtttctctagtcatctgctcatacaagtgtgaaatgatggtaccagcttggccttcttcattgcagccttctttgtaacagcagaacatcccgtgaaagggaactgttaagtacgtgaccctatctccatgtgacaaatccacacagccttgaaagggcggcattccctgacctatccaggtggggtggggggtgagggctgcagatctctatatcattatgtaaaacaatctcaaagacagagttaaattaaacagcaggtgctgagagcgtgctggacactcgttccaaacagcagcgttactgccccctagaggacattttttgaaatttatgcggccactgtcatttctggttgtcacaagactggggcttctggcatttgatagatggcaggtcagggaagctaaatgccctgtaaggtgtaggacagtctgacacaatagggaatttccaacaacaagaacacatctctcaatttccttctgtctttctgggcaattacatacatgaggaaatgtttgcaattctcagacctggaaaatggttctgttttacccatatacccacacctttttttttttttttagacagggtctctcactctgtcgctggttggagtgcagtagcacgatcacagctcactacagcctcgacctccccggctcaagcgatcctcccacctcagcctcccgagtagcacactcctctgcctgactaattcgtttgtatttgttgtagagacagggtttcgctatgctgcccaggttgtaaccatacttcttttgaatacttatattctgctatgcattgagttttccaggaataaaaccactctgcagttgtatggaaaattgggttttgtcttcaagaactttaccgaaggtcatttgccatttcattttttgtttggtttgttttgtttttttgagatggagttttgctcttgtcgcccaggctggagtgcagtggtacgatcttggctcactgtaacctctgcctcctgggttcaagcaattctcctgcctcagcctcccaagtacacgcccagctaattttgtatttttagtagagacagggtttcctcatgttggtcaggctggtctcaaactcccgacctcaggtaatctgtctgcctcggcctcccaaagtgctgggattacaggtgtgagccaccgtgcctggcctttttttttttttttttttttaagacgggacctcactgttgctcaggctggagtgcagtggtgtgatcatagctcactgcagcattgaatccctgagctcaagtgatcctagtgcttcaccttctcaagtagctgggactacaggagcgtgccacctcgcctggctaatacatatatatatatatttttttgagtcagggtctctcactctgttgcccaggctggagtgccgtggcacaatcacagctcgctgcagcctcgacgtcctggattaaagcgatcctcccacctcagctctctcccccctccccccaccagtagctgggaccacagtcacgcaccaccacgcccagctaatttttgtttattttttatagagaaggggtgtcagtatgttgcccacgatggtctcaaactcctgggctcatgtgatcctcctgcatccacctcccaaagtgtcaggattacaccacagggcccagccttagtcatttcactgtaagatgggaataataattgtactacctcaaaggattgtgttacagattaaagagcataatacagaacctggcacaatataaatctcaataaacagtaacaggacctaccttttaaaaacactgaaatgaagtatgccagctatcatcctagcagaagagattgaattaaagtccccttatatgtatttgagtacagatttctctgctaattcagagaattaggggaccttgtctccatcctgggtctcagtttcttcatctttaaaatggaaaactgggtgtggtggctcacgcctataatcccagcactttgggagcccaacgtgggcagatcacctgaggccaggagttcgagaccagcctgatcaacatggtgaaaccccatctctactaaaaatgcaaaaattagctgggcgtggtggtgcacacctgtaatcccagctacttgggaggctgaggcacaagaatcgcttgaacctgagaggcagagggtgcagtgagctgaaatcacaccactgcactccaacctgggcaacagagcgattctctgtctcaaaaataaataaataaccaaattaaaataaaaaataaaataaaattgaaaggatagtactcctggccccacttccttcctagggctgccaaggtctaagctgagagatgaggggtgtggcaacagtgcatgacagggaaagctggcctggggcgggccgctccttctcccttcctgtcctccgtgcagctgacttctccctgcccttctctgggttctggagatgactctgacccaacctctctgagctctctgagtggggcagcttcctcacctaggccctggcctattgcttcagagacaggtccccaccgggttaacaagaggaacccctttgatgtcctaaaaagaattcctcctgtttttaatgtgccagtttacaagatctcttcacatccatcatcccgtttgaacctcacatgactgtgtggagggcaagcgggggttattgtgtacacattttacagatgaggaaaccgagtcttggaggagttgagtaatccattcaagtcagttggagcagacatctcctgactcacagggggcccacctggagaaaggccaggggcactcaggtgggctggcagggctgggagggctgggagggaggatcagggccgtgagctgagctggcatcggcatccagtggagctggtgaggtgggcaaaactctgggctctgattgatttgcactcagacaccaacaccactgcatcctagctaatgactggataagactttagcatctcatatgcctgtgaatggagatcattctctggagtgttacaggaactctgcatttgaaacacaggcctgattcttgccatctctactcctgctcccagctctcacagccagagtcaacagtgcccgcctgtccttccactgccaccacccaaactgtctacctgctgcccttaacaacagataggatccaactcctagagtccccaagcaattcagggaccactcagaggtgaaacaaccccaaccattctcaaccttagcaaaaggaacagggtggtgaagggtactccccatgcccatagggacacaggacactggccagaccaccaagtggcgcctggccccccatgggccttcctgacccggtagccacccctcccaggggaaggacacagtgttctcagatgtgggcccaggggctgtgtcatggaggagctgggaagggtttagaggacctgggtttctgtctttggtttttttttttctttccactctagtaaaatcaaattttatatcattgattttgtaaagtttaattggaaaatgaatttctttgtgttgaataagtgcaatttgctggtcaaaattcattcaaacataggagtgagagaggaggaagtgactgtttattgaggaactgtgatgtgccaggcactccacaagatgttttgctcattcaggcctcctgagcaccctgggagacaggcattccaatgagcccatttcacagatgaaggcatttagggtcaaaacaataagccaaagtcacccagctggcaggaggccacgctggaatccaagctcagcagacagtgaagaggcccgtttgctctgctcccagcctgtctgctaggcttgcagaaaagcactgactcttgtcctctggcacctgccacctgcctgtctggggtgggaaaggcgagaggacaagggtgctgctttttgttataatttgattaaaatgcatttgtggccgggcgcggtggctcatgcctgtgatcccagcactttgggaggctgaggcaggcggatcgcttgagctcaagagtttgagacctgcctgggcaacatagtgaaaccctgtctctaccaaaaatacaaaaaagtagccaggcgtgttggtgtgtacctgtggtcccagctacttaggaagctgaggcagggggatcgcttgaacctggaaggtggaggttgcagtgagctgagatcgcgccactgcactccagcctgggtgagcgagaccgtgtctcaaaaaaaaaaaaaaaaagtatttgcatatgacactttgttaaaacagtgccatggctcacgcttgtaatcccagcattttaggaggatcacttgagcccaggagttcgagaccagcttgggcaatacaaccagaccccatctctaccaaaaaataaaataaaattagtcggacacggtggtgtgcacctgtagtcccagctactctggggacttaagtgggaggatcaattgagctcaggagttcaaggctgcagtgagccatgatcataccactgtactccagcttaggtgacagagtaagaccctgtgtctaaaaagcaaacaaacaaaaaacaaacagttattgagtgaaaaacaaaacagagaagtggcttatccactgtcactgggcaagttcacggcttatgctgggatgggaacatgggtctcctgactgtggtcagtacttctgttcccaaataccctgccacagcctctagcctgggcactgggctgttcctgcttctcctccctcattacccccccatagcccagtaaccccccacccctagctctggtttccctttctgcctaaagggagacgctctgtcctgatatggagcattccccccaggacccccagccctaccactcatgagttggaatagttccgaaaggacatagcctgtctgctcctgctatctctgcctgccgtgtggggagctggtccagaatgatggagcccctggggagcaaggggtcgtgtccagcttgggagggcttctggagacctggcccgagtcccagatccttcttgaactagaaaaatgcctgctgtttttagtgcattctcagttgacaaagcattttcttacctctaatcccattggatctttataatgaagaaggaattccctgtcaatctgcattgtattttatttaattaatttatttattggttttgagacagagtctcgctctgttgcccaggctggagtgcggtggcatgatctcgcctcactgcaacctccgcctcctggttcaagcaattctagtgcctcagcctctcaagtagctgggattataggggtgtgccaccacacctggctaatttttttaaaaaatttacttatttattttgagacggaatcttgctctgtcgcccagactagagtgcagtggcgtgatctcagctcactgtaacctccatctcctgggttcaactgattctctcacctcagcttcccgagtagctgggattacaggcgcccgccaccatgcccggctaatttttgtatttttagtagagatgaggtttcaccatgttagccaggctgctatcgaactcctgacctcaaatgatctggctgcctcagcccccctgcattgcattttaaatctgagagacagaggctgagagtggcctgtgtctagcaccaggacaggctacacagtttggaggtccagtgcaaatgaaaatgtgggtctccttgttcaaaagtattaggactctggccaggtgcgatggctcacgcctgtaatctcagcactttgggaggctgaggcggatggatcacctgagatcaggagtttgagaccagcctggccaatgtggtgaaagcccgtctctactaaaaatacaaaaattagctgggcatggtggcagatgcctgtaatctcagctactcaggaggctgaggcaggagaagcacttgaacccaggaggcggaggttgcagtgaaccgagatcacgccattgcactccagcctgggtgacatataaggtccagccctacggggcttagcaggtgttctccccgtgtgcggagatgagagatcataagaaataaagacacaagacaaagagataaagagaaaacagctggaccctggggaccactaccaccaagacgcggagaccggtagtggccccgaatggctgggggcactgacatctattgcatacaagacaaggggggcagggtaaggagggtgagtcgtccaagcgattgataaggtcaagcaagtcaagtgatcatgggatagggggcccttcccttttaggtagccaaagcagagagggaaggcagcatatgtcagcgttttcttctatgcatgtatcaaaaagatcaaagactttaaggctttcactatttcttctaccactatctactacgaacttcaaagaggaatccaggagtatgggaggaacatgaaagtggacaaggagcgtgaccactgaagcacagcaccacagaaaggggtttaggccttgggatgactgcgggcaggcctggataatatccagcctcccacaagaagctggcggagcagagtgtttcctgactcctcgaaggaaaggagactccctttcttggtctgctaagtagcgggtgccttcccaggcactggcgttaccgcttgaccaaggagccctcaagcggcccttatgagggcgtgacagagggctcacctcttgccttcttggtcacttctcataatgtcccttcagcacctgaccctataccctccggttattccttggttatattagtaatacaacaaagagtcatattaaaggctaataattaataatgtctatactaatgattgataatgcccatgaccatctctatatctaatttgtattataactattctcattctaactattttctttattatactgaaacagtctgtgccttcaatctcttgcctcggcacctgggtaatcctccgcccacagtgacaagagtgaaactctgtcttaaaaaaaaaaattaagaatttcaagataggacagcagagctttaaattttttttttttcctagaggcagagtcttgctctcttgcccaggctggggtgcattgttgcaatcacatatcactgcagccttgagctcccgggctcaagtggtcatcctgctttggcctcccaaagtcctgggattacaggcgtgagccactgcacccggcctgtagagctttaaacaaagcacaggctgcttctgagcacagggccctcgtcacatggctatgaagccagccctgcctggacccagaaccctggtgtgggcactttatagcttataagccctccagtcaagaggcctcagcctgagcctccctgagcctcagtttcctctcctgtaatctgtgaaagcactgagcactaatctcccttccgggctagctttctgcaatagcttaaagctcccatcccaagatctctccaagaatcctcttaccaagtccctgctcccagcctggctccctggtccccatccctctcccctctcacaccctttcaaagcccagaaaggacaggtgaccgagtgactgtctctgggctcatacagtggggtggtctcctgtgacaggctagggcttcagggtatggcttagcctatccctcacacttccttggggcctcttggggcagacagcccaggcggggcccagcaggcacactagtcaggtgcctgatgatgagggcaggcgcctggaactgggtcaacaatcgctcacacggaggtgactcaagcagcaaggggaaaaagaggtgggggtggggaggtggctgtttctgtgccctcacttgagcctcagcttttagggttgccagatttatttttatttttatttttgagagtctcactctgtctcccaggctggagtgcagtggcgcaatcttggctcactgcaacctctgcctcccaggttcaagcaattctcgtgcctcagcctcccaagtagctgggattacagatgtgcaccaccacccctggctaatttttgtatttttagtagagatggggtttcaccatgttggccagtctggttttgaactcctaacctcaggtgatccacccacctcagcctcccaaagtgctgggattataggcgtgagccaccattcctggcagcctgaaaattttttaatttttttttaatttttttttttttttagtgatggggtctccatatgttgcccaggctggtctcaaactcctaggctcaagtgatcctttcaccttggccttggcctcctaaagtgctgggattacaggcgtgacccactgtgcctgcctgctggtatgtaacttgttgttttttttttttttttagacaaagtctcacttgccctggctcaagtgcagtggtgccatcttggctcactgcagtctccgcctcccaggttcaagtgattctcctccctcagcctcctgagtagctgggattacaggcatgtgccaccatgcccagctaatttttgtatttttagtagagacagggtttcaccatgctggccaggctggtcttgaactcctaaccttgagtgatctacccacctctgcctcccaaagtgctggattacaggccaataggcctggccccctgtaacttttttttttttttttaatttttgagacagagtcttgctctgtcgcccaggctggagtgcagtggtgccatctcggctcactgcaagctccgcctccctggttcacgccattctcctgcctcagcctcccgagtagctgggactacaggcgcctaccaccacgcctggctaattttttggatttttagtagagatggagtttcaccgtgttagccaggatggtctccatttcctgaccttgtgatccacccgcctcagcctcccaaagtgctgggattacaggcgtgagccaccacgcccggcccctgtaacttcttaagcgaaggaaaataaaaaaaaaaaaaaagcagaagccacaaaggaaaagattgaccaatatatttcagatgacataacaacaacacaaaagacaaatgacagattgggaggaagcatctgaaacatacaaaatacccagaatatataaacagctcctacagttcaacaagaaagaaaatactgcaaaaacaatcagcaaataatactgtgatggtagaacaatgtatctacttggatggtgctcctggattagattaacatttaaatcagtgaactttggttaaagcaaattgctctccattgtagggagaccccctgaaactattgctacggaataaaagatgaaatgctcctgattattgtaaatacgaaattgcatgcaggattgtgtaaagacaatgccaggttggactgccagtataagccaacagctcgtgatgtgcttccccctgaagagagcctatgaacagacgtgcagtcagggaggtttcacatcaccaatattcctatcccagaaaagcagatgttcatagccctgggaatggaatgtgacccttgtggagggcctataaatggacgcatgagcggcacctgttcatatggataagatagggctataaatgccctcatcttgccatggctcttctaggcctctttaaggttaaggcatactcccttctgataatttctggtctaactggttatctagcttcacgtcctgtttctatggattgtttgtaaccagcttttgctgcaactcttactgctgattaatatcttgctaatcataggttatggaaagactgtgtttctgttttaaggctctgttagaaattactgatgcacacactatattgtaaattcttatctctgtatactgtacttctgcatacagatgttatgttaaagaattacttcatccccatgtgaccatctcacctcataatcaaacgaccctaaatccctcactaacctaccctcgccctcactaaacttaataataaatgctggtatatccagtgcattggcagcatcgcaggaccagaaggcggtgacccccttggacccagctttcactatcttgtgtgtgtctattatttcccgacctgccgatccacctggggacaaagagagagccccattgccttgtgggctgctggccagatcccgcgatactccataatgtggatggacctgaataaaacagaaagacgggcctcgccgaactagagggaattctcctgcaggctaccctcagacttaaccatcagctctcctgggtcgccagcccaccagctcacactaaaggcttaggatggttatgataagaaagacagatgataacagaagttaaccatggatgaggctaaagagaaattgaaaccctcatacatcgctggtgggaatgtaaaatagtgcagctgctttggaaaacaggctggtagttacacaaaaggctaaacagaattattatgtaatccactaattctactcctaggtatatacccaagagaaatgaaaacatgtccacacaaaaacttgtatgggccgggcgtggtggctcactcctgtaatcccagccctttgggaggctgaagtgggaggatccctggagttcaggagtttgagactagctgaccaacatggcaagaccctgtctctacaaaaaccacacaaaaattagctggacatggtgataagcacctgtagtcccagctacttgggaggcggaggtgggaggattacttgagcccaggaggcagaggttgcagtgagcagagatgacaccactgcactccaacctgggtaacagagcaataccctgtctcaaaaaaaaaaaaaaaaatgctggggggatgatgaagataggcacagtggctcaagcctgtaatctgtaatctcagcattttgggaggctgaggcagcaggatcacttgaggccaggagcttgagaccagactaggcaacatattaagacaaaaaaatacaaaatatagctgggagtggtggcatgtgtctgtattcccagctactcaggaggctgaggtgggagggtctgaggctgcagtacaaggtggcaccactgtaatccagcttgggcggcagaggaaggctctgtctcaaaaacaaagaaacaaacaaaaatgggccacgcgtggtggctcacgcctgtaatcccagcagtctgggagattgaggtgggtggatcacttgaggtcaggagttcgagaccagcctggtcaacatggcggaaccctgtctctactaaaatacaaaaattagctgggcatcatggcacatgcttgtaatcccagctatccgggaggctgagacaggagaattgcttgaacccaggaggcagaggttgcagtgagccaagatcgtgccactgcactccagcttgagtgttggagtgagactctgtctcaaacaaatgaaaagaaacaaacaaaaaagaaatgagatactgatacctgctatgtgataagctttgaaaacattatgctaagtgaaagaagccagttgcagatcacatattataaaatttcatttatatggaatatccaaatctatagaggtagaaagtagatcagtggttgcctagggttagggagggtgactgctaaaggacataaggctgccttctgtggtgataaaaatggtcttaaactgattgtaatgatgctgcataactctcttaagacactaaaaaccattgagttgtacactttaatttttttgtgttggcgggggtggatggagtctcgctctgttgtccaggctggaatgcagtggcacgatctcggtttactgcaacctctgcatcccgggttcaagcgattctcctgcctcggcctcctgagtagggaattacagacctcgttatcgtggcaccttacccttctgatgttaaaaaaaaaaaaaaaaagagcgagagagagagagagaaacatttgtgaagtaggttgttgagtctcagcactattgaccttttgggcaggatacttctttgttgtgggggattgttctgtgtgtcgtgtgatgtttagtgggattgctggcccttacctaccagatgccagtgtccctccaccctgagttgtgacaacccagattgtctccagacactcctaaatgtccctggccggcaaaattgccgctgctcaagaatcacggctttgacgattagactttgtgatatttgtttcagtctgtttaggttttttttcttctacctgtatttttttctggttctgggtggttgtaattagtaggttattgatcgattcacctaacatttcatgaaagtttcatgtgtgtgtgtgtttcaatagaagcataaactatactccctagtctcaagatacacaggaaggaaaataagcacaaatgtgtcaccagggcacagactagtactaggtcctcagcaggccaggtgtcttatccgctgtctgggtctgctctagctccaggcttagaacccctgccacacgactccacagctcggttggcaccctttccctcctccgacttctgctgcctcgagcttggttagccatccccctgcccctgcctcatcctcagctccagttccttgctcaggctgcagcagtctccatcccctgtgcagacactgccgttcctccacggcccagtatcaggctttccctgggcctctcctctctcctggcccatctcccatcatccatctctgcctggcccaggccctttggcaccaagcaggctgactcttgtcactggctaatctgttctgtggtacattttctctcctcaccctcccatatcaattcctcgaaggcagggccgatctggagactaggaagccacttctctttcgacagcccccaccacagcccagcccgtgccaggcacccagcagctcctgaagcccactggcattgaacatggcattcaatccctgccaagcctgcccttcccatctggtttcccagggctcttcccaacacctcctcctccacctgccagttaaaatcttcccagactcagctcaaggagatgctcctaaggtggaatgaaatctcttcttccccacctggagacaatctacttcctctccctacacctggcaactggcgcacaaccttgtatcttaaattagattcagcctgagactgtctcccaccaatccctgctccctgtcctgctgagcaccttgaggaaagggctttggggctgtttatctttgtcctggaaaccatccttcaactcactctggggcctgcctagcatgtcaaccgagtttggagaatagggcagaatagggcaggacaggacaggacaagacagggcaggataggataggagcgagccagctcagtagctcacatttgtaatcccagcgccttggggggctgcggtaggagaatcgctttgggagcaggagttgcaggccgcagtgagctatgatcagcttgggcgactgagcgagaccctgtctctaaaacaaacacacaagtccgggcgcggtggctcatgcctgtaatcttagcactttgggaggccgaggtgggcggatcacgaggtcaagaaatcgagaccatcctggccaacatggtgaaaccccgtctctactaaaaatacaaaaattagctgggcgtggtggtgcgcgcctgtagtcccagctactcgggaggctgaggcaggagaatcgcttgaacccgggaggcagaggttgcagtgagccgagatcgtgccactgcactccagcctggcgacagagtgagactccgtctcagaacaaacaaacaaaaggatagaaaggcgagcacaaatattcccaattcataacactccctcgcactgtcaatgccccagacacgcgctatcatctctagcaaactcccccaggcgcctgcaggatgggttaaggaaggcgacgagcaccagctgccctgctgaggctgtcccgacgtcacatgattctccaatcacatgatccctagaaatggggtgtggggcgagaggaagcagggaggagagtgatttgagtagaaaagaaacacagcattccaggctggccccacctctatattgataagtagccaatgggagcgggtagccctgatccctggccaatggaaactgaggtaggcgggtcatcgcgctggggtctgtagtctgagcgctacccggttgctgctgcccaaggaccgcggagtcggacgcaggtaggagagcggccgcgcagacctctcgcctgctcctgcccaggggcccgccagggccatgtgagcttgaggttcccctggagtctcagccggagacaacagaagaaccgcttactgaaactccttgggggttctgatacactag

SEQ ID NO: 77 - DNA, 2630 bp, Artificial Sequence
  Synthetic polynucleotide
gaggtagaaagtagatcagtggttgcctagggttagggagggtgactgctaaaggacataaggctgccttctgtggtgataaaaatggtcttaaactgattgtaatgatgctgcataactctcttaagacactaaaaaccattgagttgtacactttaatttttttgtgttggcgggggtggatggagtctcgctctgttgtccaggctggaatgcagtggcacgatctcggtttactgcaacctctgcatcccgggttcaagcgattctcctgcctcggcctcctgagtagggaattacagacctcgttatcgtggcaccttacccttctgatgttaaaaaaaaaaaaaaaaagagcgagagagagagagagaaacatttgtgaagtaggttgttgagtctcagcactattgaccttttgggcaggatacttctttgttgtgggggattgttctgtgtgtcgtgtgatgtttagtgggattgctggcccttacctaccagatgccagtgtccctccaccctgagttgtgacaacccagattgtctccagacactcctaaatgtccctggccggcaaaattgccgctgctcaagaatcacggctttgacgattagactttgtgatatttgtttcagtctgtttaggttttttttcttctacctgtatttttttctggttctgggtggttgtaattagtaggttattgatcgattcacctaacatttcatgaaagtttcatgtgtgtgtgtgtttcaatagaagcataaactatactccctagtctcaagatacacaggaaggaaaataagcacaaatgtgtcaccagggcacagactagtactaggtcctcagcaggccaggtgtcttatccgctgtctgggtctgctctagctccaggcttagaacccctgccacacgactccacagctcggttggcaccctttccctcctccgacttctgctgcctcgagcttggttagccatccccctgcccctgcctcatcctcagctccagttccttgctcaggctgcagcagtctccatcccctgtgcagacactgccgttcctccacggcccagtatcaggctttccctgggcctctcctctctcctggcccatctcccatcatccatctctgcctggcccaggccctttggcaccaagcaggctgactcttgtcactggctaatctgttctgtggtacattttctctcctcaccctcccatatcaattcctcgaaggcagggccgatctggagactaggaagccacttctctttcgacagcccccaccacagcccagcccgtgccaggcacccagcagctcctgaagcccactggcattgaacatggcattcaatccctgccaagcctgcccttcccatctggtttcccagggctcttcccaacacctcctcctccacctgccagttaaaatcttcccagactcagctcaaggagatgctcctaaggtggaatgaaatctcttcttccccacctggagacaatctacttcctctccctacacctggcaactggcgcacaaccttgtatcttaaattagattcagcctgagactgtctcccaccaatccctgctccctgtcctgctgagcaccttgaggaaagggctttggggctgtttatctttgtcctggaaaccatccttcaactcactctggggcctgcctagcatgtcaaccgagtttggagaatagggcagaatagggcaggacaggacaggacaagacagggcaggataggataggagcgagccagctcagtagctcacatttgtaatcccagcgccttggggggctgcggtaggagaatcgctttgggagcaggagttgcaggccgcagtgagctatgatcagcttgggcgactgagcgagaccctgtctctaaaacaaacacacaagtccgggcgcggtggctcatgcctgtaatcttagcactttgggaggccgaggtgggcggatcacgaggtcaagaaatcgagaccatcctggccaacatggtgaaaccccgtctctactaaaaatacaaaaattagctgggcgtggtggtgcgcgcctgtagtcccagctactcgggaggctgaggcaggagaatcgcttgaacccgggaggcagaggttgcagtgagccgagatcgtgccactgcactccagcctggcgacagagtgagactccgtctcagaacaaacaaacaaaaggatagaaaggcgagcacaaatattcccaattcataacactccctcgcactgtcaatgccccagacacgcgctatcatctctagcaaactcccccaggcgcctgcaggatgggttaaggaaggcgacgagcaccagctgccctgctgaggctgtcccgacgtcacatgattctccaatcacatgatccctagaaatggggtgtggggcgagaggaagcagggaggagagtgatttgagtagaaaagaaacacagcattccaggctggccccacctctatattgataagtagccaatgggagcgggtagccctgatccctggccaatggaaactgaggtaggcgggtcatcgcgctggggtctgtagtctgagcgctacccggttgctgctgcccaaggaccgcggagtcgga

SEQ ID NO: 78 - DNA, 1200 bp, Artificial Sequence
  Synthetic polynucleotide
tcaatccctgccaagcctgcccttcccatctggtttcccagggctcttcccaacacctcctcctccacctgccagttaaaatcttcccagactcagctcaaggagatgctcctaaggtggaatgaaatctcttcttccccacctggagacaatctacttcctctccctacacctggcaactggcgcacaaccttgtatcttaaattagattcagcctgagactgtctcccaccaatccctgctccctgtcctgctgagcaccttgaggaaagggctttggggctgtttatctttgtcctggaaaccatccttcaactcactctggggcctgcctagcatgtcaaccgagtttggagaatagggcagaatagggcaggacaggacaggacaagacagggcaggataggataggagcgagccagctcagtagctcacatttgtaatcccagcgccttggggggctgcggtaggagaatcgctttgggagcaggagttgcaggccgcagtgagctatgatcagcttgggcgactgagcgagaccctgtctctaaaacaaacacacaagtccgggcgcggtggctcatgcctgtaatcttagcactttgggaggccgaggtgggcggatcacgaggtcaagaaatcgagaccatcctggccaacatggtgaaaccccgtctctactaaaaatacaaaaattagctgggcgtggtggtgcgcgcctgtagtcccagctactcgggaggctgaggcaggagaatcgcttgaacccgggaggcagaggttgcagtgagccgagatcgtgccactgcactccagcctggcgacagagtgagactccgtctcagaacaaacaaacaaaaggatagaaaggcgagcacaaatattcccaattcataacactccctcgcactgtcaatgccccagacacgcgctatcatctctagcaaactcccccaggcgcctgcaggatgggttaaggaaggcgacgagcaccagctgccctgctgaggctgtcccgacgtcacatgattctccaatcacatgatccctagaaatggggtgtggggcgagaggaagcagggaggagagtgatttgagtagaaaagaaacacagcattccaggctggccccacctctatattgataagtagccaatgggagcgggtagccctgatccctggccaatggaaactgaggtag

SEQ ID NO: 79 - DNA, 21 bp, Artificial Sequence
  Synthetic polynucleotide
cguucgcacaaccuuguautt

SEQ ID NO: 80 - DNA, 21 bp, Artificial Sequence
  Synthetic polynucleotide
cuggcgcacaaccuuugcutt

SEQ ID NO: 81 - DNA, 21 bp, Artificial Sequence
  Synthetic polynucleotide
cguucgcacaaccuuugcutt

SEQ ID NO: 82 - DNA, 21 bp, Artificial Sequence
  Synthetic polynucleotide
auacaagguugugcgaacgtt

SEQ ID NO: 83 - DNA, 21 bp, Artificial Sequence
  Synthetic polynucleotide
agcaaagguugugcgccagtt

SEQ ID NO: 84 - DNA, 21 bp, Artificial Sequence
  Synthetic polynucleotide
agcaaagguugugcgaacgtt